(19) United States
(12) Patent Application Publication
(10) Pub. No.: US 2012/0116183 A1
(43) Pub. Date: May 10, 2012

(54) CLASSIFYING SEIZURES AS EPILEPTIC OR NON-EPILEPTIC USING EXTRA-CEREBRAL BODY DATA

(76) Inventor: Ivan Osorio, Leawood, KS (US)

(21) Appl. No.: 13/288,886

(22) Filed: Nov. 3, 2011

Related U.S. Application Data
(63) Continuation-in-part of application No. 13/098,262, filed on Apr. 29, 2011.
Application No. 13/098,262 is a continuation-in-part of application No. 12/896,525, filed on Oct. 1, 2010.

(51) Int. Cl. A61B 5/00; A61B 5/01; A61B 5/145; A61N 1/08; A61B 5/11

(52) U.S. Cl. 600/301; 607/45; 600/300; 600/595; 600/309

(57) ABSTRACT

A method of distinguishing a non-epileptic seizure from an epileptic seizure in a patient, comprising: detecting a seizure in a patient based on at least one first body signal of the patient selected from an autonomic signal, a neurologic signal, a metabolic signal, an endocrine signal, and a tissue stress marker signal; analyzing at least one second body signal of the patient selected from an autonomic signal, a neurologic signal, a metabolic signal, an endocrine signal, and a tissue stress marker signal; determining, based on the analyzing, at least a first classification index comprising at least one of an epileptic seizure index and a non-epileptic seizure index; and classifying the seizure as a non-epileptic seizure or an epileptic seizure based on the at least a first classification index. A medical device system capable of implementing the method. A computer-readable device for storing data that, when executed, perform the method.

The present application is a Continuation-in-Part of U.S. patent application Ser. No. 13/098,262, filed Apr. 29, 2011, now pending, which is a Continuation-in-Part of U.S. patent application Ser. No. 12/896,525, filed Oct. 1, 2010, now pending. Both U.S. patent application Ser. Nos. 13/098,262 and 12/896,525 are hereby incorporated by reference in their entirety.

### BACKGROUND OF THE INVENTION

1. Field of the Invention

This invention relates to medical device systems and methods capable of classifying an occurring or impending seizure as epileptic or non-epileptic using extra-cerebral body data.

2. Description of the Related Art

Non-epileptic generalized seizures, also known as pseudo-seizures, psychogenic seizures, or hysterical seizures, are often misdiagnosed as epileptic at large cost to the patient, caregivers, and the health care system. The diagnosis of non-epileptic seizures is difficult, as evidenced by the long mean latency (7 years) between the onset of manifestations and accurate diagnosis that the patient's seizures are non-epileptic. Approximately 10-30% of patients referred to epilepsy centers because of suspected epileptic seizures are diagnosed as having non-epileptic seizures. While carrying the incorrect diagnosis of epileptic seizures, patients are treated with anti-seizure medications. Since these lack efficacy (due to the fundamental pathophysiologic differences between epileptic and non-epileptic seizures), emergency room visits and hospitalizations are frequent. The observation that the prevalence of non-epileptic seizures is higher in patients with epilepsy (estimates put the number of patients having both epileptic and non-epileptic seizures at 10-50% of all epileptic patients seen at specialty centers) than in the general population, makes accurate differentiation between them even more challenging.

Video-EEG monitoring, the current “gold standard” for differentiation, requires hospitalization, usually for several days, at high expense to the health care system and great inconvenience to the patient and his/her loved ones.

### SUMMARY OF THE INVENTION

In one embodiment, the present disclosure provides a method of distinguishing a non-epileptic seizure from an epileptic seizure in a patient, comprising: detecting a seizure in a patient based on at least one first body signal of the patient selected from an autonomic signal, a neurologic signal, a metabolic signal, an endocrine signal, and a tissue stress marker signal; analyzing at least one second body signal of the patient selected from an autonomic signal, a neurologic signal, a metabolic signal, an endocrine signal, and a tissue stress marker signal; determining, based on the analyzing of the at least one second body signal, at least a first classification index comprising at least one of an epileptic seizure index and a non-epileptic seizure index; and classifying the seizure as one of a non-epileptic seizure or an epileptic seizure based on the at least a first classification index.

In one embodiment, the present disclosure provides a method of distinguishing an epileptic seizure from a non-epileptic seizure, comprising: identifying an unclassified seizure that is one of an epileptic seizure or a non-epileptic seizure; determining a first seizure classification index having an index class selected from a neurologic index class, an autonomic index class, a motor index class, a tissue stress marker index class, or a metabolic index class; determining a second seizure classification index having an index class selected from a neurologic index class, an autonomic index class, a motor index class, a tissue stress marker index class, or a metabolic index class; classifying said seizure as one of an epileptic seizure or a non-epileptic seizure based on both said first and said second seizure classification indices; and taking at least one further action based on said classifying, wherein said at least one further action is selected from:

issuing a notification that the seizure is non-epileptic; issuing a notification that the seizure is epileptic; administering a therapy for a non-epileptic seizure; administering a therapy for an epileptic seizure; or logging at least one of whether the seizure is an epileptic or non-epileptic seizure and at least one of the date of the seizure, the time of occurrence of the seizure, the severity of the seizure, the time elapsed from a previous seizure, or the frequency per unit time of the same type of seizure.

In one embodiment, the present disclosure provides a method, comprising: receiving a kinetic signal from at least one target of the patient's body; determining at least one kinetic index based on said kinetic signal; identifying an unclassified seizure based on the at least one kinetic index; receiving at least one of a non-kinetic neurologic index and an autonomic index; and classifying the seizure as an epileptic seizure or non-epileptic seizure based on the at least one of a non-kinetic neurologic index and an autonomic index.

In one embodiment, the present disclosure provides a medical device system, comprising: at least one sensor configured to receive at least one of an autonomic signal indicative of an autonomic activity of a patient, a neurologic signal indicative of a neurologic activity of said patient, a metabolic signal indicative of a metabolic activity of said patient, an endocrine signal indicative of an endocrine activity of said patient, or a tissue stress marker signal indicative of a tissue stress marker activity of said patient; a seizure detection unit configured to detect a seizure in a patient based on said at least one autonomic, neurologic, metabolic, endocrine, or tissue stress marker signal; at least one classification index determination unit configured to determine at least a first classification index selected from an autonomic index, a neurologic index, a metabolic index, an endocrine index, and a tissue stress marker index; and a seizure classification unit configured to classify said epileptic seizure as one of an epileptic seizure and a non-epileptic seizure based at least in part on said at least a first classification index.

In one embodiment, the present disclosure pro-vides a non-transitive, computer-readable storage device for storing data that when executed by a processor, perform a method as described above.

### BRIEF DESCRIPTION OF THE DRAWINGS

The invention may be understood by reference to the following description taken in conjunction with the accompanying drawings, in which like reference numerals identify like elements, and in which:

FIG. 1 provides a stylized diagram of a medical device for classifying a seizure as epileptic or non-epileptic, in accordance with one illustrative embodiment of the present disclosure;

FIG. 2 provides a block diagram of a medical device system that includes a medical device and a monitoring unit, in accordance with one illustrative embodiment of the present disclosure;

FIG. 3A provides a block diagram of an autonomic signal module of a medical device, in accordance with one illustrative embodiment of the present disclosure;

FIG. 3B provides a block diagram of a neurologic signal module of a medical device, in accordance with one illustrative embodiment of the present disclosure;

FIG. 3C provides a block diagram of a metabolic signal module of a medical device, in accordance with one illustrative embodiment of the present disclosure;

FIG. 3D provides a block diagram of an endocrine signal module of a medical device, in accordance with one illustrative embodiment of the present disclosure;

FIG. 3E provides a block diagram of a stress marker signal module of a medical device, in accordance with one illustrative embodiment of the present disclosure;

FIG. 3F provides a block diagram of a classification unit of a medical device, in accordance with one illustrative embodiment of the present disclosure;

FIG. 4 shows a flowchart depiction of a method, in accordance with one illustrative embodiment of the present disclosure;

FIG. 5 shows a flowchart depiction of a method, in accordance with one illustrative embodiment of the present disclosure;

FIG. 6 shows a flowchart depiction of a method, in accordance with one illustrative embodiment of the present disclosure; and

FIG. 7 compares simulated, non-limiting (a) epileptic and (b) non-epileptic movements of an arm, as represented by the path traced by an accelerometer, as seen from in front of, to the side of, and above a patient.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

This document does not intend to distinguish between components that differ in name but not function. In the following discussion and in the claims, the terms “including” and “includes” are used in an open-ended fashion, and thus should be interpreted to mean “including, but not limited to.” Also, the term “couple” or “couples” is intended to mean either a direct or an indirect electrical connection. “Direct contact,” “direct attachment,” or providing a “direct coupling” indicates that a surface of a first element contacts the surface of a second element with no substantial attenuating medium there between. The presence of small quantities of substances, such as bodily fluids, that do not substantially attenuate electrical connections does not vitiate direct contact. The word “or” is used in the inclusive sense (i.e., “and/or”) unless a specific use to the contrary is explicitly stated.

The term “electrode” or “electrodes” described herein may refer to one or more stimulation electrodes (i.e., electrodes for delivering a therapeutic signal generated by an MD to a tissue), sensing electrodes (i.e., electrodes for sensing a physiological indication of a state of a patient's body), and/or electrodes that are capable of delivering a therapeutic signal, as well as performing a sensing function.

Identification of changes in brain state (whether physiologic or pathologic) has traditionally been accomplished through analysis of electrical brain signals and behavioral observation. Continuous (e.g., round-the-clock) automated monitoring of changes in brain state imposes certain limitations on the utilization of these traditional methods, due to the difficulties inherent to automated ambulatory video, the large amount of data produced per unit time, and the excessive demands on human and technical resources required to maintain an acceptable signal/noise for electrical signals recorded from the scalp. Additionally, scalp signals have poor temporo-spatial resolution, a characteristic which results in both low sensitivity and specificity of state-of-brain detection changes.

Implanted sensors or electrodes beneath the scalp but above the outer skull table or intra-cranial (epidural, subdural or depth) have been used to overcome the limitations of scalp recordings. However, although the quality of recordings (especially for intracranial electrodes) is much better (e.g., typically has a higher S/N) than that from scalp electrodes, the quality is still limited and there are risks (e.g., infection, bleeding, brain damage) associated with these devices, not to mention cost and scarcity of neurosurgeons to perform this type of procedures.

While electrical brain signals and behavioral observation may provide information for classification of brain states, this task can be accomplished more efficiently, and/or more cost-effectively through monitoring of other biological signals under control of the brain such those generated by the heart, muscle, skin, eyes, tympanic membrane temperature, and body posture/movement, since they may not require surgery, or if surgery is required for implantation, the procedures are much shorter, simpler, and cheaper that those required for recording of brain signals and there is no shortage of human resources.

Certain highly valuable neurological signals (e.g., cognitive) for detection, quantification, and classification of state changes may obtained non-invasively and can be used in this invention.

In one aspect, the present invention is directed to differentiating between generalized or convulsive epileptic and non-epileptic seizures. In another aspect, extra-cerebral signals may be also used to detect and distinguish partial epileptic from partial non-epileptic seizures.

Extra-cerebral signals (e.g., EKG, body movements, respirations, etc.) denote herein body signals that may be recorded/obtained without placing sensors or electrodes on the head (e.g., scalp or under it) or inside the head/skull (e.g., subdural electrodes/sensors, depth or intra-cerebral electrodes/sensors) and are directly or indirectly under control of the central or peripheral nervous system. Cognitive signals such as attention/responsiveness, language, memory among others, while directly generated by the brain are considered extra-cerebral herein, since they may be recorded/obtained without resorting to sensors/electrodes placed on/inside the head. Extra-cerebral signals provide valuable and reliable information about the state of the brain since they are either generated by the brain or under its control.

Aspects of extra-cerebral signals such as magnitude, rate of change (including return to non-seizure levels), and morphology (e.g., waveform), vary depending on whether the event affecting the extra-cerebral signal(s) is epileptic or non-epileptic. The present invention discloses methods and apparatus for using such signals to distinguish between these seizure types. For example, although movements, cardiac and respiratory rate increase during both epileptic and non-epileptic seizures, the type, magnitude and duration of changes (much different and greater with epileptic seizures) can be used, according to the present disclosure, for accurate classification of the seizure as epileptic or non-epileptic.

Although in many embodiments discussed herein, at least one extra-cerebral signal may be used for differentiation of non-epileptic seizures from epileptic ones, nothing in this disclosure precludes the use of cerebral signals for said purpose.

These extra-cerebral (e.g., autonomic, neurologic, etc.) signals can be used individually or in combination to monitor continuously the brain and generate a state-of the-system/organ report, in real-time for the detection, quantification, classification, validation, control and logging of physiologic or pathologic state changes. This approach takes advantage of the inherent and finely tuned dynamical coupling among these systems. For instance, changes in brain state/activity may result in changes in heart activity, muscle activity, and skin properties.

Herein, Applicant describes a method, systems, and devices that may: a) detect in real-time pre-specified changes in brain state; b) quantify their duration, intensity, extent of spread, and time of occurrence; c) classify their type (e.g., epileptic vs. non-epileptic seizures; primarily vs. secondarily generalized seizures; generalized vs. partial seizures; complex vs., simple partial seizures; d) use as a basis for warning and control/therapy, and/or e) save this information to memory for future retrieval for optimization of detection, quantification and classification of state changes and assessment and optimization of therapeutic (e.g., control) efficacy. Non-epileptic movements in this invention refer to those resembling movements seen during tonic-clonic seizures but which are not caused by those abnormal electrical activity that characterizes epileptic seizures.

In embodiments where extra-cerebral data is the basis for a detection, cerebral signals, such as EEG/ECoG, evoked potentials, field potentials, or single unit activity, among others, may be used for validation, confirmation, or the like of the extra-cerebral detections or determinations made from extra-cerebral data.

The extra-cerebral epileptic and/or non-epileptic event detection disclosed herein provides a comprehensive, cost-effective, valuable alternative to systems of epileptic or non-epileptic event detection based on brain electrical signals such as EEG. To date, no extra-cerebral systems for detection of both epileptic and non-epileptic seizures have been developed or commercialized. Extra-cerebral epileptic and non-epileptic event detection may make use of one or more signals of autonomic, neurologic, endocrine, metabolic, gastro-intestinal, and/or dermal origin and of tissue/organ stress markers, such as those presented in Table 1.

Extra-cerebral detection of state changes takes advantage of the fact that certain brain structures directly or indirectly influence autonomic, endocrine, gastro-intestinal, dermal and metabolic functions and that certain abnormal states (e.g. seizures) stress the body tissues and result in the elevation of certain compounds or molecules (e.g., stress markers) that may be used to detect, quantify and verify the occurrence of said abnormal state.

It has been established that seizures in humans originating from or spreading to central autonomic structures induce changes in heart rate, among other cardio-vascular indices, and also in respiratory activity. It should be stated that seizure-induced heart rate or respiratory increases (which are far more frequent than heart rate or respiratory rate decreases) are not primarily the result of increased motor activity or of metabolic changes, but are instead largely a neurogenic phenomenon.

In the present disclosure, a highly robust, efficient and reliable system is provided for detecting, quantifying and/or classifying epileptic and/or non-epileptic seizures based upon at least one extra-cerebral signal and, if desired, using this information to provide warnings, or notification, of the type of seizure event (epileptic or non-epileptic) detected. In some embodiments, systems of the present invention may take appropriate therapeutic interventions that reflect (for efficacy and safety purposes) their etiologic and patho-physiologic differences. Systems of the present disclosure are suitable for long-term implantable or external devices, and may provide reliable and accurate indications of seizure events for a wide variety of epilepsy and/or non-epilepsy patients.

### Extra-Cerebral Multimodal Signals (Table 1) Autonomic

Cardiac: EKG, PKG, Echocardiography, Apexcardiography (ApKG), Intra-cardiac pressure, Cardiac blood flow, cardiac thermography; from which can be derived, e.g., heart rate (HR), change of HR, rate of change of HR, heart rate variability (HRV), change of HRV, rate of change of HRV, HRV vs. HR. Also, blood pressure, heart sounds, heart rhythm, heartbeat wave morphology, heartbeat complex morphology, and thoracic wall deflection.
Vascular: Arterial Pressure, Arterial and venous blood wave pressure morphology; Arterial and venous blood flow velocity, arterial and venous blood flow sounds, arterial and venous thermography
Respiratory: Frequency, tidal volume, minute volume, respiratory wave morphology, respiratory sounds, end-tidal CO2, Intercostal EMG, Diaphragmatic EMG, chest wall and abdominal wall motion, from which can be derived, e.g., respiration rate (RR), change of RR, rate of change of RR. Also, arterial gas concentrations, including oxygen saturation, as well as blood pH can be considered respiratory signals.
Other autonomic: Skin resistance, skin temperature, skin blood flow, sweat gland activity, body temperature, organ temperature.
Concentrations of catecholamines (and their metabolites) and acetylcholine or acetylcholinesterase activity in blood, saliva and other body fluids concentrations and its rate of change.

### Neurologic

Cognitive/behavioral: Level of consciousness, attention, reaction time, memory, visuo-spatial, language, reasoning, judgment, mathematical calculations, auditory, and/or visual discrimination.
Kinetic: Direction, speed/acceleration, trajectory (1D to 3D), pattern, and quality of movements, force of contraction, body posture, body orientation/position, body part orientation/position in reference to each other and to imaginary axes, muscle tone, agonist-to-antagonist muscle tone relation, from which can be derived, e.g., information about gait, posture, normal or abnormal head, ocular, trunk, pelvis, and limb movements and falls.
Vocalizations: Formed, unformed, rate of production/unit time, pitch, loudness, duration.

### Endocrine

Prolactin, luteinizing hormone, follicle stimulation hormone, growth hormone, ACTH, cortisol, vasopressin, beta-endorphin, beta, lipotropin-, corticotropin-releasing factor (CRF).

### Tissue Stress Markers

CK, lactic acid, troponin, neuron-specific enolase, reactive oxygen and nitrogen species including but not limited to iso- and neuro-prostanes and nitrite/nitrate ratio, glutathione, glutathione disulfide and glutathione peroxidase activity, citrulline, protein carbonyls, thiobarbituric acid, the heat shock protein family, catecholamines, lactic acid, N-acetylaspartate, and metabolites of any of the foregoing.

### Metabolic

Arterial pH and gases, lactate/pyruvate ratio, electrolytes, glucose, oxygen consumption.

Certain body signals may fall within more than one class. For example, pH is listed in Table 1 as both an autonomic signal, since it is under respiratory control, and a metabolic signal, since it is also influenced by metabolic by-products.

Generally, when the term “reference value” is used herein without further qualification, it refers to a value of an index determined from autonomic, neurologic, endocrine, metabolic, or stress marker data and derived from an interictal, ictal or postictal period of an epileptic or of a non-epileptic seizure. The evolution of non-epileptic seizures at long temporal scales (e.g., days to years) behaves similarly to that of epileptic seizures, having “interictal” periods when no abnormal activity occurs, “ictal” periods when the abnormal activity is visible (e.g., “loss of consciousness and generalized abnormal motor activity) and “postictal” periods (e.g., the abnormal movements cease but the patient remains “unresponsive”). Reference values or ranges thereof for any of the autonomic, neurologic, endocrine, metabolic or stress marker features may be dependent upon the patient's level of consciousness (e.g., wakefulness), level of physical (e.g., exercise) or cognitive activity (e.g., attentive), time of day (e.g., circadian), or seizure history (e.g., mean or median time between seizures, seizure severity, etc., over short-term or long-term time periods) and are thus non-stationary. Although reference values for a certain feature in a certain patient state and/or time are most directly comparable to corresponding signals in the same patient state and/or time of day, they may be comparable to corresponding signals from other states, times, or both.

A reference value may be a mean, median, or another statistical indicator of the central tendency of a data series or of its distribution (e.g. probability distribution or density function). The reference value may be a threshold that is useful—depending upon the underlying index or feature being considered—for distinguishing between an epileptic (ictal) state and a non-ictal state, and also for distinguishing a convulsive/generalized epileptic from a convulsive/generalized non-epileptic state. Body movements of several body parts occur during both convulsive/generalized epileptic and non-epileptic seizures but differences in velocity, force direction and number of planes which the movement traverses, may be used to distinguish epileptic from non-epileptic seizures.

The reference value may be from a data series taken from the same patient on whom the present methods may be performed, or a plurality of data series taken from each member of a population of patients. If the reference value is derived from the patient's own data, it is desirable for the patient to have an epileptic and/or a non-epileptic seizure history, in order to identify activity that is indicative of an epileptic and/or non-epileptic seizure, and thereby to obtain data that may be used as reference value for comparison purposes. The reference value may be empirical in origin or derived from accepted wisdom in the field.

The various embodiments disclosed by parent U.S. patent application Ser. Nos. 13/098,262 and 12/896,525 may be also used to distinguish epileptic generalized from psychogenic non-epileptic generalized seizures whose kinetic activity, but not patho-physiology, resembles that of epileptic seizures.

An extra-cerebral signal approach relying on multiple body signals such as kinetic, autonomic and metabolic signals, and that may be performed in ambulatory patients (unlike video-EEG monitoring, the current “gold standard” that requires hospitalization for several days) is ideally suited for classifying seizures as either epileptic or non-epileptic, given the high sensitivity and specificity inherent to a multimodal “extra-cerebral” signal approach according to the disclosure described herein. Embodiments of the present invention may be implemented in small portable devices, which would provide a cost-effective solution (no hospital admission would be necessary) to a pressing medical need.

The following are a few examples of features of epileptic and/or non-epileptic seizures, one or more of which may be used to distinguish between epileptic generalized/convulsive seizures and non-epileptic generalized/convulsive seizures: a) The intensity of non-epileptic movements, unlike that of epileptic movements, waxes and wanes (crescendo-decrescendo pattern) throughout the event; b) Non-epileptic movements, unlike epileptic movements, are multi-directional or multi-planar, said changes in direction or plane occurring rapidly and often in a random sequence. For example, vertical movements may give way to horizontal movements, and these in turn to oblique or rotary or flapping movements; c) Body joints movements in non-epileptic seizures, unlike in epileptic seizures, are incoherent or disorganized: e.g., while the right upper extremity is moving in the vertical plane at a certain speed and with certain amplitude and phase, the direction, speed, phase and amplitude of movement of the left upper extremity at the same time may be different; d) in non-epileptic seizures, unlike in epileptic seizures, co-activation of agonist and antagonist muscle groups is rarely seen: Co-activation of the abdominal and paraspinal muscles during an epileptic generalized tonic-clonic seizure keeps the torso straight while the sole activation of paraspinal muscles, a common observable in non-epileptic generalized seizures, manifests as an arched back (opisthotonus); unopposed activation of neck, trunk, hip flexors results in the so-called “fetal position”, also a common occurrence in non-epileptic seizures; e) Involvement (in the form of movements) of certain body parts is commonly found in non-epileptic seizures while they are rarely if ever seen in epileptic generalized seizures; pelvic thrust, pelvic gyrations, and other pelvic movements are nearly pathognomic of non-epileptic seizures; f) Metabolic (lactic) acidosis occurs with epileptic generalized tonic-clonic seizures and not with non-epileptic generalized seizures; g) oxygen desaturation and carbon dioxide retention are seen in epileptic convulsive but not in non-epileptic convulsive seizures.

Although not limited to the following, an exemplary system capable of implementing embodiments of the present disclosure is described below.

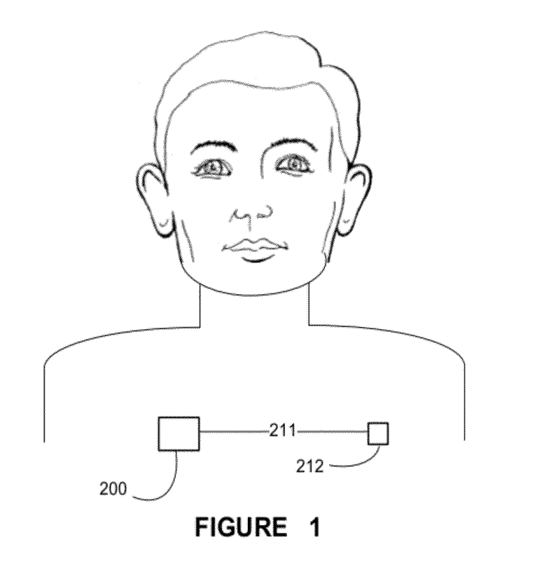

Turning now to FIG. 1, a stylized medical device system is depicted. The medical device system comprises a medical device 200 and at least one sensor 212.

In some embodiments, the medical device 200 may be implantable, while in other embodiments, such as that shown in FIG. 1, the medical device 200 may be completely external to the body of the patient. In still other embodiments, the present invention may comprise systems with some implantable portions and some external portions.

The sensor 212 may be implanted in the patient's body, worn external to the patient's body, or positioned in proximity to but not in contact with the patient's body. The sensor 212 may be configured to receive cardiac activity data, body movement data, responsiveness data, awareness data, or other data from the patient's body.

FIG. 1 depicts the medical device 200 being in wired communication 211 with the at least one sensor 212. In other embodiments (not shown), the medical device 200 may be in wireless communication with the at least one sensor 212.

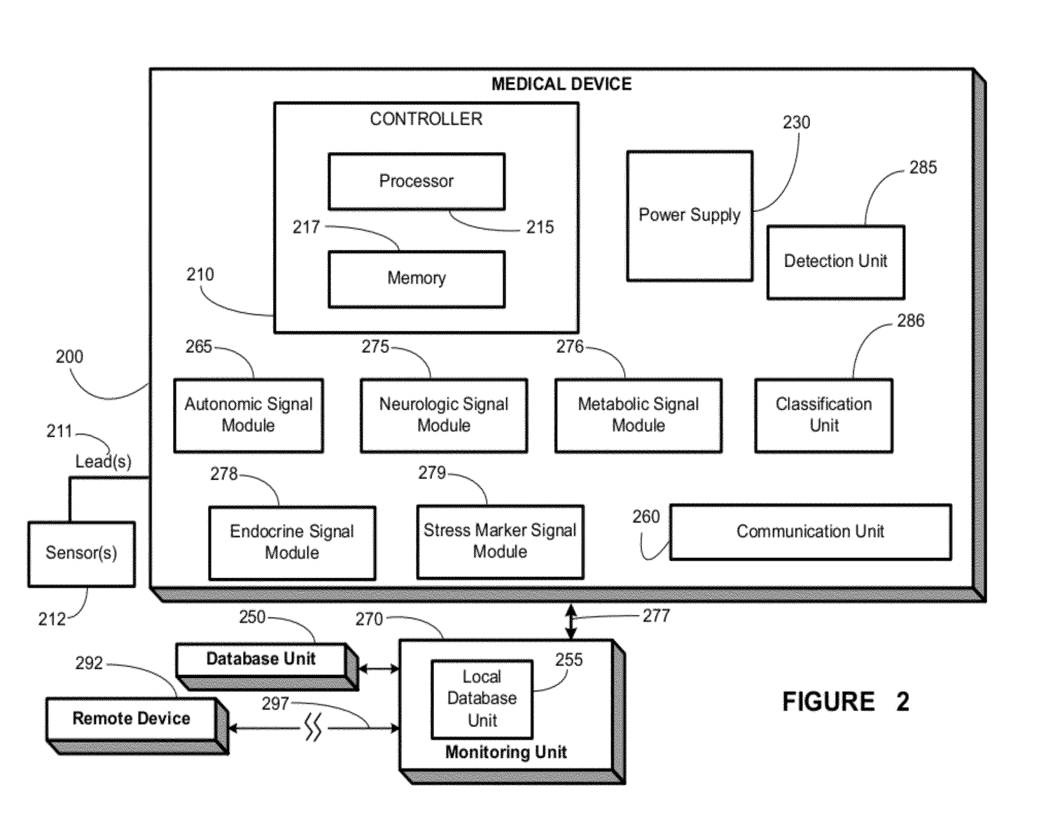

Turning now to FIG. 2, a block diagram depiction of a system comprising a medical device 200 is provided, in accordance with one illustrative embodiment of the present disclosure. In some embodiments, the medical device 200 may be implantable, while in other embodiments the medical device 200 may be completely external to the body of the patient. In still other embodiments, the medical device may include both implanted and non-implanted portions. FIG. 2 illustrates various components, units and/or modules that perform functions discussed more fully below. It will be appreciated that these components and units or modules may be equivalently described using similar terms, and the use of particular terms herein is not intended to exclude embodiments involving different components performing the same function, or which may be described using different but similar terms.

The medical device 200 may comprise a controller 210 capable of controlling various aspects of the operation of the medical device 200. The controller 210 is capable of receiving internal data or external data and is capable of affecting substantially all functions of the medical device 200.

The controller 210 may comprise various components, such as a processor 215, a memory 217, etc. The processor 215 may comprise one or more microcontrollers, microprocessors, etc., capable of performing various executions of software components. The memory 217 may comprise various memory portions where a number of types of data (e.g., internal data, external data instructions, software codes, status data, diagnostic data, etc.) may be stored. In one embodiment, the memory 217 may be configured to store at least one reference value of an autonomic activity, a neurologic activity, a metabolic activity, an endocrine activity, or a stress marker activity. The memory 217 may comprise one or more of random access memory (RAM), dynamic random access memory (DRAM), electrically erasable programmable read-only memory (EEPROM), flash memory, etc.

The medical device 200 may also comprise a power supply 230. The power supply 230 may comprise a battery, voltage regulators, capacitors, etc., to provide power for the operation of the medical device 200, including delivering the therapeutic electrical signal. The power supply 230 comprises a power source that in some embodiments may be rechargeable. In other embodiments, a non-rechargeable power source may be used. The power supply 230 provides power for the operation of the medical device 200, including electronic operations and the electrical signal generation and delivery functions. The power supply 230 may comprise a lithium/thionyl chloride cell or a lithium/carbon monofluoride (LiCFx) cell if the medical device 200 is implantable, or may comprise conventional watch or 9V batteries for external (i.e., non-implantable) embodiments. Other battery types known in the art of medical devices may also be used.

The medical device 200 may also comprise a communication unit 260 capable of facilitating communications between the medical device 200 and various devices. In particular, the communication unit 260 is capable of providing transmission and reception of electronic signals to and from a monitoring unit 270, such as a handheld computer or PDA that can communicate with the medical device 200 wirelessly or by cable. The communication unit 260 may include hardware, software, firmware, or any combination thereof.

The medical device 200 may also comprise one or more sensor(s) 212 coupled via sensor lead(s) 211 to the medical device 200 (or in wireless communication with the medical device 200, not shown). The sensor(s) 212 are capable of receiving signals related to a physiological parameter, such as an autonomic signal, a neurologic signal, a metabolic signal, an endocrine signal, or a stress marker signal, among others, and delivering the signals to the medical device 200. The at least one sensor(s) 212, in one embodiment, may comprise an accelerometer. The at least one sensor(s) 212, in another embodiment, may comprise an inclinometer. In another embodiment, the at least one sensor(s) 212 may comprise an actigraph. One or more of the at least one sensor(s) 212 may be external structures that may be placed on the patient's skin, such as over the patient's heart or elsewhere on the patient's torso. The at least one sensor(s) 212, in one embodiment, may comprise a multimodal signal sensor capable of detecting various signals, such as autonomic and neurologic signals.

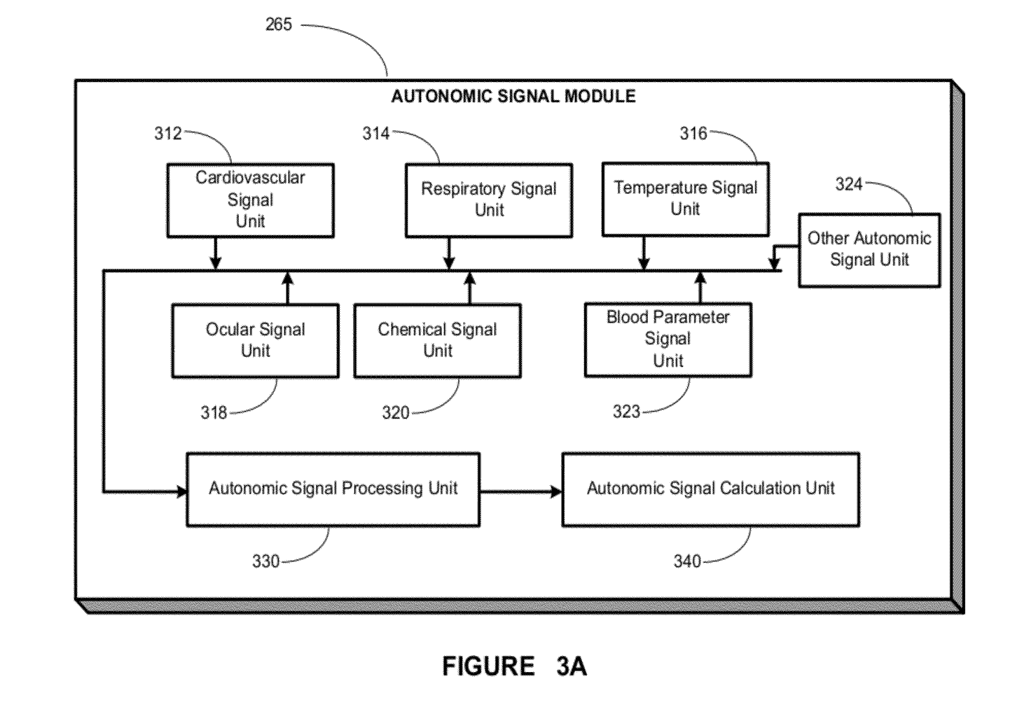

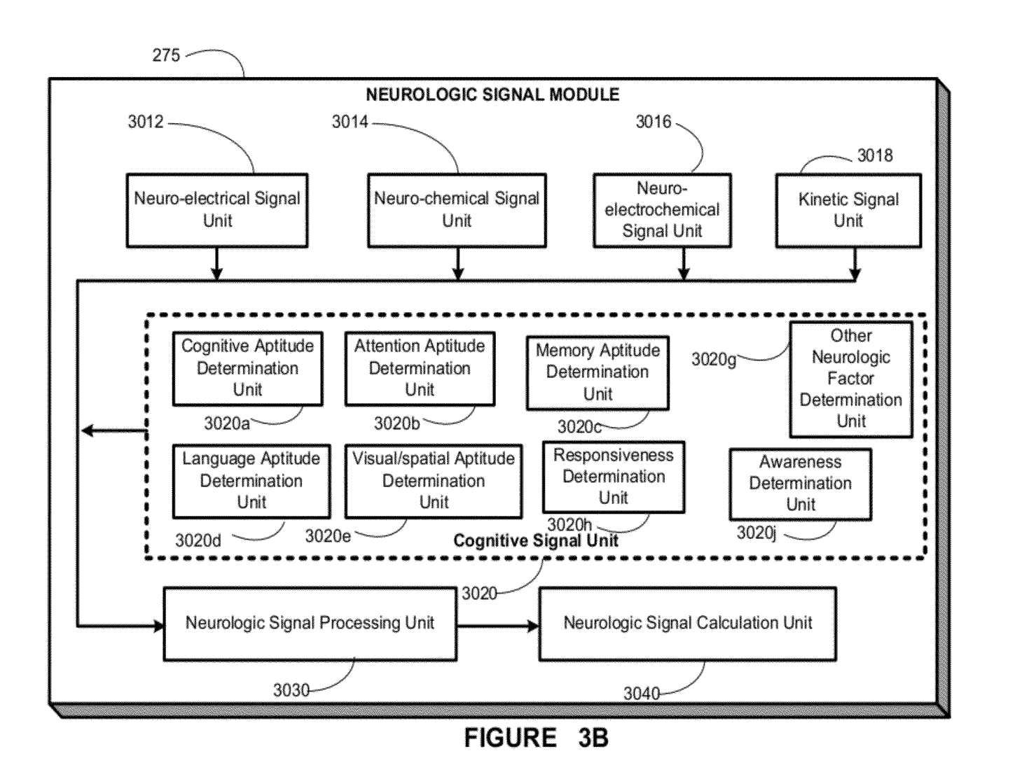

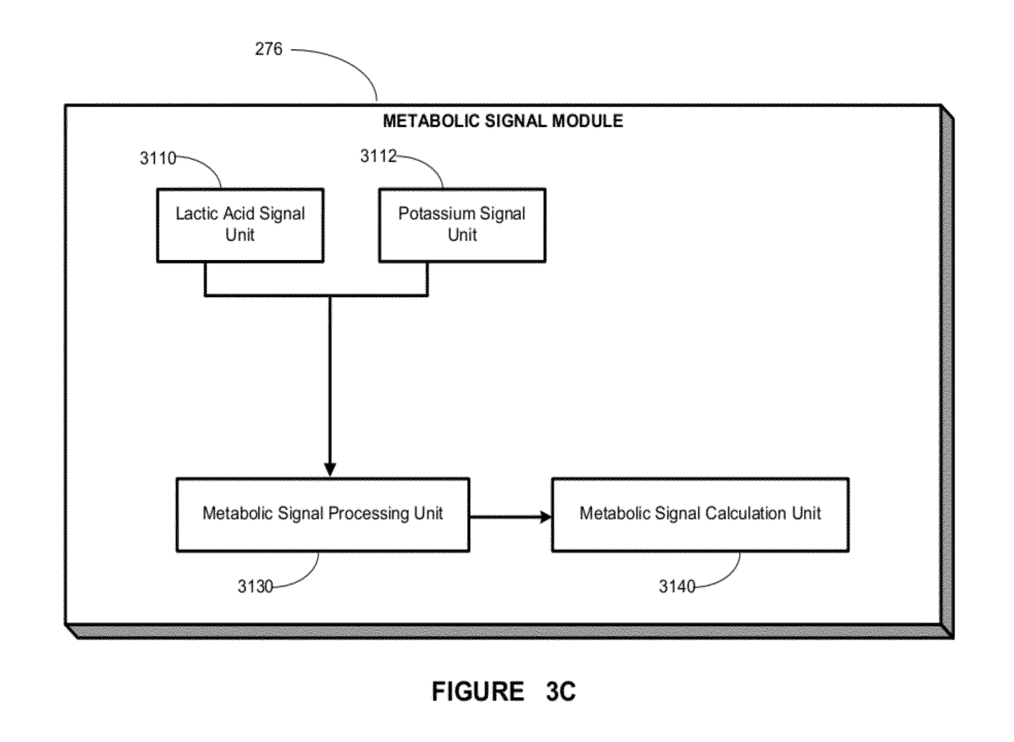

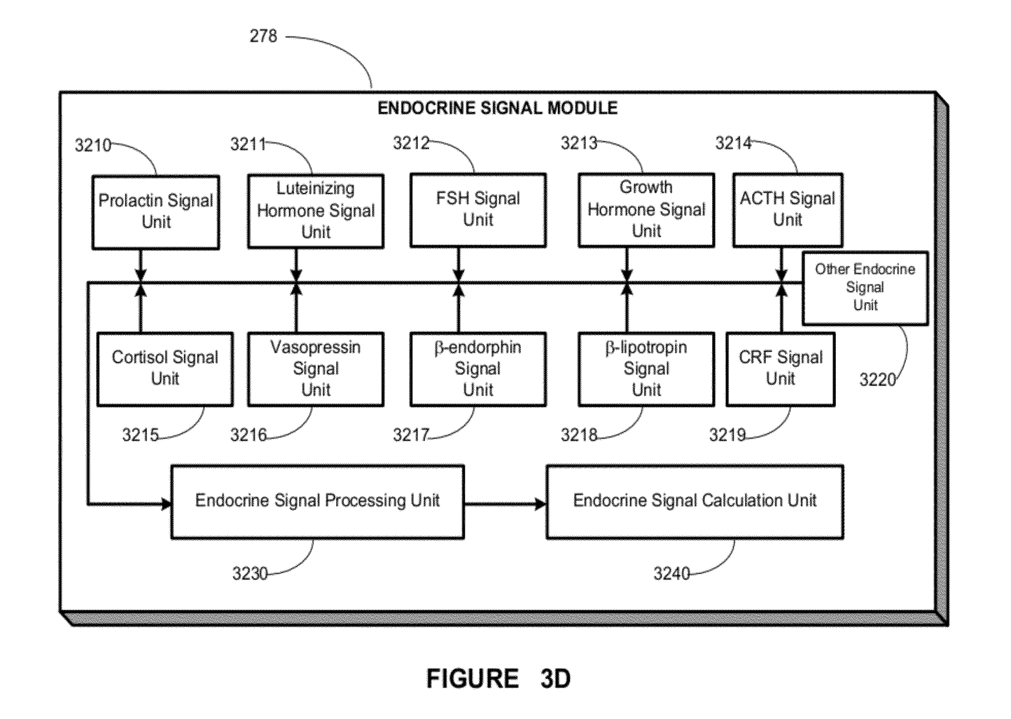

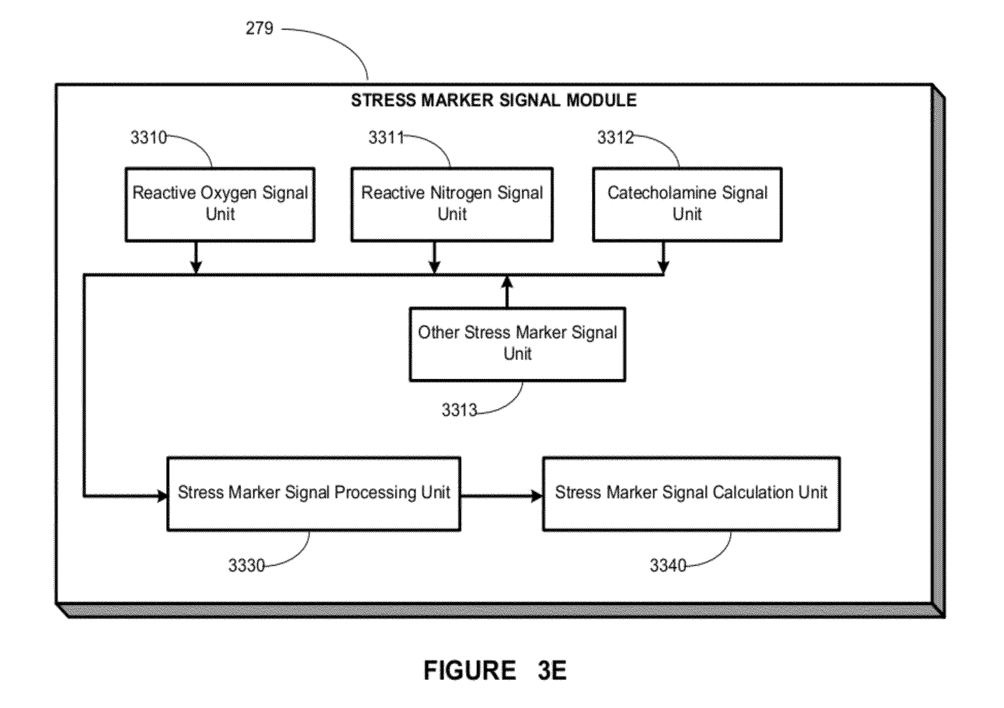

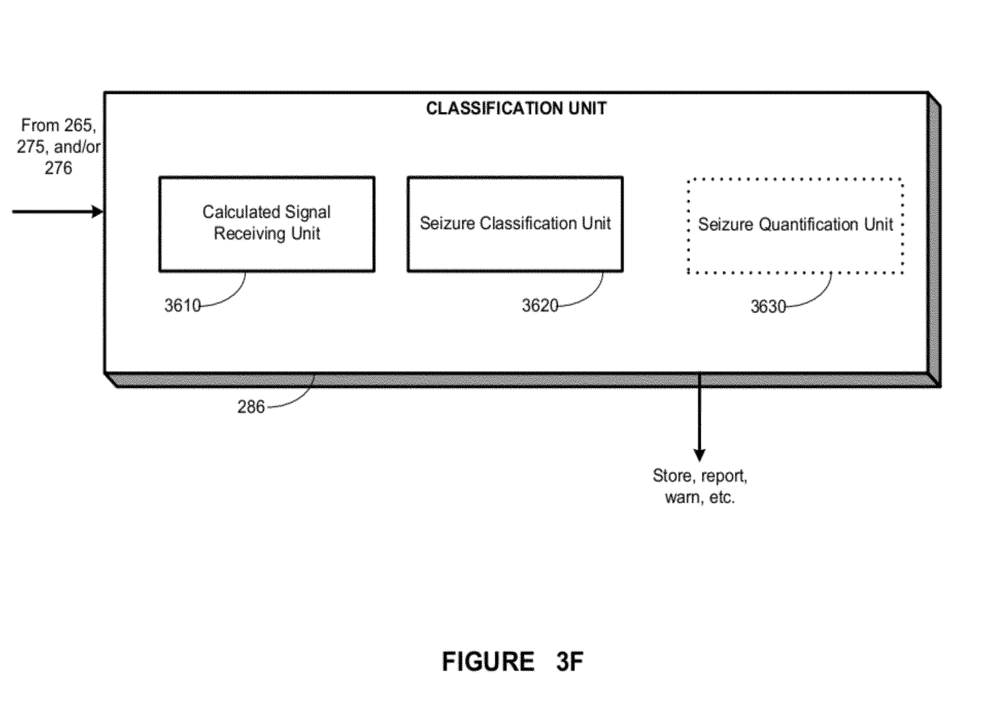

In one embodiment, the medical device 200 may comprise a autonomic signal module 265 that is capable of collecting autonomic data, e.g., cardiac data comprising fiducial time markers of each of a plurality of heart beats, a blood SaO2 value, a blood CO2 concentration, a blood pH value, a body temperature, or an infrared activity of a portion of a patient's body, among others. The autonomic signal module 265 may also process or condition the autonomic data. The autonomic data may be provided by the sensor(s) 212. The autonomic signal module 265 may be capable of performing any necessary or suitable amplifying, filtering, and performing analog-to-digital (A/D) conversions to prepare the signals for downstream processing. The autonomic data module 265, in one embodiment, may comprise software module(s) that are capable of performing various interface functions, filtering functions, etc. In another embodiment, the autonomic signal module 265 may comprise hardware circuitry that is capable of performing these functions. In yet another embodiment, the autonomic signal module 265 may comprise hardware, firmware, software and/or any combination thereof. A more detailed illustration of the autonomic signal module 265 is provided in FIG. 3A and accompanying description below.

The autonomic signal module 265 is capable of collecting autonomic data and providing the collected autonomic data to a detection unit 285, a classification unit 286, or both.

In one embodiment, the medical device 200 may comprise a neurological signal module 275 that is capable of collecting neurologic data, e.g., signals indicative of a motor activity of the patient, such as a crescendo-decrescendo pattern of a motor activity, a force of a motor activity, a velocity of a motor activity, a multi-directionality of a motor activity, a multi-planarity of a motor activity, a frequency of a motor activity, an incoherence or asymmetry between a first motor activity and a second motor activity, a lack of coactivation during certain motor activity of an agonist muscle group and an antagonist muscle group, or a pelvic motor activity, among others. (“Lack” and “absence” may be used interchangeably herein). The neurological signal module 275 may also process or condition the neurologic data. The neurologic data may be provided by the sensor(s) 212. The neurological signal module 275 may be capable of performing any necessary or suitable amplifying, filtering, and performing analog-to-digital (A/D) conversions to prepare the signals for downstream processing. The neurological signal module 275, in one embodiment, may comprise software module(s) that are capable of performing various interface functions, filtering functions, etc. In another embodiment, the neurological signal module 275 may comprise hardware circuitry that is capable of performing these functions. In yet another embodiment, the neurological signal module 275 may comprise hardware, firmware, software and/or any combination thereof. Further description of the neurologic signal module 275 is provided in FIG. 3B and accompanying description below.

The neurological signal module 275 is capable of collecting neurologic data and providing the collected neurologic data to a detection unit 285, a classification unit 286, or both.

In one embodiment, the medical device 200 may comprise a metabolic signal module 276 that is capable of collecting metabolic data, e.g., signals indicative of a metabolic activity of the patient, such as a lactic acid concentration or a potassium concentration, among others. The metabolic signal module 276 may also process or condition the metabolic data. The metabolic data may be provided by the sensor(s) 212. The metabolic signal module 276 may be capable of performing any necessary or suitable amplifying, filtering, and performing analog-to-digital (A/D) conversions to prepare the signals for downstream processing. The metabolic signal module 276, in one embodiment, may comprise software module(s) that are capable of performing various interface functions, filtering functions, etc. In another embodiment, the metabolic signal module 276 may comprise hardware circuitry that is capable of performing these functions. In yet another embodiment, the metabolic signal module 276 may comprise hardware, firmware, software and/or any combination thereof. Further description of the metabolic signal module 276 is provided in FIG. 3C and accompanying description below.

The metabolic signal module 276 is capable of collecting metabolic data and providing the collected metabolic data to a detection unit 285, a classification unit 286, or both.

In one embodiment, the medical device 200 may comprise an endocrine signal module 278 that is capable of collecting endocrine data, e.g., signals indicative of an endocrine activity of the patient, such as a prolactin concentration, a luteinizing hormone concentration, a follicle-stimulating hormone (FSH) concentration, a growth hormone concentration, an ACTH concentration, a cortisol concentration, a vasopressin concentration, a β-endorphin concentration, a β-lipotropin concentration, or a CRF concentration, among others. The endocrine signal module 278 may also process or condition the endocrine data. The endocrine data may be provided by the sensor(s) 212. The endocrine signal module 278 may be capable of performing any necessary or suitable amplifying, filtering, and performing analog-to-digital (A/D) conversions to prepare the signals for downstream processing. The endocrine signal module 278, in one embodiment, may comprise software module(s) that are capable of performing various interface functions, filtering functions, etc. In another embodiment, the endocrine signal module 278 may comprise hardware circuitry that is capable of performing these functions. In yet another embodiment, the endocrine signal module 278 may comprise hardware, firmware, software and/or any combination thereof. Further description of the endocrine signal module 278 is provided in FIG. 3D and accompanying description below.

The endocrine signal module 278 is capable of collecting endocrine data and providing the collected endocrine data to a detection unit 285, a classification unit 286, or both.

In one embodiment, the medical device 200 may comprise a stress marker signal module 279 that is capable of collecting stress marker data, e.g., signals indicative of a stress marker activity of the patient, such as a reactive oxygen concentration, a reactive nitrogen concentration, or a catecholamine concentration, among others. The stress marker signal module 279 may also process or condition the stress marker data. The stress marker data may be provided by the sensor(s) 212. The stress marker signal module 279 may be capable of performing any necessary or suitable amplifying, filtering, and performing analog-to-digital (A/D) conversions to prepare the signals for downstream processing. The stress marker signal module 279, in one embodiment, may comprise software module(s) that are capable of performing various interface functions, filtering functions, etc. In another embodiment, the stress marker signal module 279 may comprise hardware circuitry that is capable of performing these functions. In yet another embodiment, the stress marker signal module 279 may comprise hardware, firmware, software and/or any combination thereof. Further description of the stress marker signal module 279 is provided in FIG. 3E and accompanying description below.

The stress marker signal module 279 is capable of collecting stress marker data and providing the collected stress marker data to a detection unit 285, a classification unit 286, or both.

The detection unit 285 may be capable of detecting a seizure. The detection unit 285 may make use of an autonomic signal provided by autonomic signal module 265, a neurological signal module 275, other modules depicted in FIG. 2, modules not shown in the figures, or two or more thereof. The detection unit 285 can implement one or more algorithms. The detection unit 285 may comprise software module(s) that are capable of performing various interface functions, filtering functions, etc. In another embodiment, the detection unit 285 may comprise hardware circuitry that is capable of performing these functions. In yet another embodiment, the detection unit 285 may comprise hardware, firmware, software and/or any combination thereof. Further description of an exemplary detection unit 285 is provided in U.S. patent application Ser. Nos. 13/098,262 and 12/896,525.

In another embodiment, the medical device 200 may further comprise a therapy unit. The therapy unit (not shown) may be configured to at least one of administer a non-epileptic seizure therapy, prevent delivery of an epilepsy therapy, or warn against delivery of an epilepsy therapy, in response to receiving from the detection unit an indication the seizure is non-epileptic. In one embodiment, the medical device 200 may further comprise a notification unit. The notification unit (not shown) may be configured to notify at least one of the patient, a caregiver, or a medical professional that the seizure is non-epileptic, based upon an indication that the seizure is non-epileptic. In one embodiment, the medical device 200 may further comprise a logging unit. The logging unit (not shown) may be configured to log at least one of the date of occurrence of the seizure, the time of occurrence of the seizure, or the severity of the seizure.

In addition to components of the medical device 200 described above, a medical device system may comprise a storage unit to store an indication of at least one of seizure or an increased risk of a seizure. The storage unit may be the memory 217 of the medical device 200, another storage unit of the medical device 200, or an external database, such as a local database unit 255 or a remote database unit 250. The medical device 200 may communicate the indication via the communications unit 260. Alternatively or in addition to an external database, the medical device 200 may be adapted to communicate the indication to at least one of a patient, a caregiver, or a healthcare provider.

In various embodiments, one or more of the units or modules described above may be located in a monitoring unit 270 or a remote device 292, with communications between that unit or module and a unit or module located in the medical device 200 taking place via communication unit 260. For example, in one embodiment, one or more of the autonomic signal module 265, the neurologic signal module 275, or the detection unit 285 may be external to the medical device 200, e.g., in a monitoring unit 270. Locating one or more of the autonomic signal module 265, the neurologic signal module 275, or the detection unit 285 outside the medical device 200 may be advantageous if the calculation(s) is/are computationally intensive, in order to reduce energy expenditure and heat generation in the medical device 200 or to expedite calculation.

The monitoring unit 270 may be a device that is capable of transmitting and receiving data to and from the medical device 200. In one embodiment, the monitoring unit 270 may be a computer system capable of executing a data-acquisition program. The monitoring unit 270 may be controlled by a healthcare provider, such as a physician, at a base station in, for example, a doctor's office. In alternative embodiments, the monitoring unit 270 may be controlled by a patient in a system providing less interactive communication with the medical device 200 than another monitoring unit 270 controlled by a healthcare provider. Whether controlled by the patient or by a healthcare provider, the monitoring unit 270 may be a computer, preferably a handheld computer or PDA, but may alternatively comprise any other device that is capable of electronic communications and programming, e.g., hand-held computer system, a PC computer system, a laptop computer system, a server, a personal digital assistant (PDA), an Apple-based computer system, a cellular telephone, etc. The monitoring unit 270 may download various parameters and program software into the medical device 200 for programming the operation of the medical device, and may also receive and upload various status conditions and other data from the medical device 200. Communications between the monitoring unit 270 and the communication unit 260 in the medical device 200 may occur via a wireless or other type of communication, represented generally by line 277 in FIG. 2. This may occur using, e.g., wand 155 (FIG. 1) to communicate by RF energy with an implantable signal generator 110. Alternatively, the wand may be omitted in some systems, e.g., systems in which the MD 200 is non-implantable, or implantable systems in which monitoring unit 270 and MD 200 operate in the MICS bandwidths.

In one embodiment, the monitoring unit 270 may comprise a local database unit 255. Optionally or alternatively, the monitoring unit 270 may also be coupled to a database unit 250, which may be separate from monitoring unit 270 (e.g., a centralized database wirelessly linked to a handheld monitoring unit 270). The database unit 250 and/or the local database unit 255 are capable of storing various patient data. These data may comprise patient parameter data acquired from a patient's body, therapy parameter data, seizure severity data, and/or therapeutic efficacy data. The database unit 250 and/or the local database unit 255 may comprise data for a plurality of patients, and may be organized and stored in a variety of manners, such as in date format, severity of disease format, etc. The database unit 250 and/or the local database unit 255 may be relational databases in one embodiment. A physician may perform various patient management functions (e.g., programming parameters for a responsive therapy and/or setting references for one or more detection parameters) using the monitoring unit 270, which may include obtaining and/or analyzing data from the medical device 200 and/or data from the database unit 250 and/or the local database unit 255. The database unit 250 and/or the local database unit 255 may store various patient data.

One or more of the blocks illustrated in the block diagram of the medical device 200 in FIG. 2 may comprise hardware units, software units, firmware units, or any combination thereof. Additionally, one or more blocks illustrated in FIG. 2 may be combined with other blocks, which may represent circuit hardware units, software algorithms, etc. Additionally, any number of the circuitry or software units associated with the various blocks illustrated in FIG. 2 may be combined into a programmable device, such as a field programmable gate array, an ASIC device, etc.

Turning to FIG. 3A, an autonomic signal module 265 is shown in more detail. The autonomic signal module 265 can comprise a cardiovascular signal unit 312 capable of providing at least one cardiovascular signal. Alternatively or in addition, the autonomic signal module 265 can comprise a respiratory signal unit 314 capable of providing at least one respiratory signal. Alternatively or in addition, the autonomic signal module 265 can comprise a blood parameter signal unit 323 capable of providing at least one blood parameter signal (e.g., blood glucose, blood pH, blood gas, SaO2 value, CO2 concentration, etc.). Alternatively or in addition, the autonomic signal module 265 can comprise a temperature signal unit 316 capable of providing at least one temperature signal. Alternatively or in addition, the autonomic signal module 265 can comprise an optic signal unit 318 capable of providing at least one optic signal. Alternatively or in addition, the autonomic signal module 265 can comprise a chemical signal unit 320 capable of providing at least one body chemical signal. Alternatively or in addition, the autonomic signal module 265 can comprise one or more other autonomic signal unit(s) 324, such as a skin resistance signal unit or an infrared activity unit configured to provide at least one signal relating to an infrared activity of a portion of a patient's body, among others.

The autonomic signal module 265 can also comprise an autonomic signal processing unit 330. The autonomic signal processing unit 330 can perform any filtering, noise reduction, amplification, or other appropriate processing of the data received by the signal units 312-324 desired prior to calculation of the autonomic signal.

The autonomic signal module 265 can also comprise an autonomic signal calculation unit 340. The autonomic signal calculation unit 340 can calculate an autonomic signal from the data passed by the autonomic signal processing unit 330. A calculated autonomic signal herein refers to any signal derivable from the provided signals, with or without processing by the autonomic signal processing unit 330.

More description regarding the autonomic signal module 265 may be found in U.S. patent application Ser. Nos. 13/098,262 and 12/896,525.

Turning to FIG. 3B, an exemplary embodiment of a neurologic signal module 275 is shown. The neurologic signal module 275 can comprise at least one of a neuro-electrical signal unit 3012 capable of providing at least one neuro-electrical signal; a neuro-chemical signal unit 3014 capable of providing at least one neuro-chemical signal; a neuro-electrochemical signal unit 3016 capable of providing at least one neuro-electrochemical signal; a kinetic signal unit 3018 capable of providing at least one kinetic signal; or a cognitive signal unit 3020 capable of providing at least one cognitive signal. The cognitive signal unit 3020 may be a component of a remote device.

In one embodiment, the cognitive signal unit comprises at least one of a cognitive aptitude determination unit 3020a capable of processing at least one cognitive aptitude indication; an attention aptitude determination unit 3020b capable of processing at least one attention aptitude indication; a memory aptitude determination unit 3020c capable of processing at least one memory indication; a language aptitude determination unit 3020d capable of processing at least one language indication; a visual/spatial aptitude determination unit 3020e capable of processing at least one visual/spatial indication; one or more other neurologic factor determination unit(s) 3020g; or a responsiveness determination unit 3020h.

The neurologic signal module 275 can also comprise a neurologic signal processing unit 3030. The neurologic signal processing unit 3030 can perform any filtering, noise reduction, amplification, or other appropriate processing of the data received by the signal units 3012-3020 desired prior to calculation of the neurologic signal.

The neurologic signal module 275 can also comprise a neurologic signal calculation unit 3040. The neurologic signal calculation unit 3040 can calculate a neurologic signal from the data passed by the neurologic signal processing unit 3030. A calculated neurologic signal herein refers to any signal derivable from the provided signals.

For example, the neurologic signal calculation unit 3040 may calculate a motor activity signal, such as a signal relating to a crescendo-decrescendo pattern of a motor activity, a force of a motor activity, a velocity of a motor activity, a multi-directionality of a motor activity, a multi-planarity of a motor activity, a frequency of a motor activity, an incoherence or asymmetry between a first motor activity and a second motor activity, a lack of coactivation during motor activity of an agonist muscle group and an antagonist muscle group, or a pelvic motor activity.

Other motor activity signal(s) that may be calculated from relevant data include those relating to the body's (or of a portion thereof such as the head, eyes, an arm, or a leg) acceleration; direction; position; smoothness, amplitude, force of movements and number of movements per unit time, and whether there are extraneous or abnormal body oscillations during resting conditions or movement. The data sources from which the signal may be calculated include electromyography, a mechanogram, an accelerometer, an actigraph, an inclinometer, or a video/optical signal, as received by kinetic capability determination unit 3018, and, optionally, further processed by neurologic data processing unit 3030.

More description regarding the neurologic signal module 275 may be found in U.S. patent application Ser. Nos. 13/098,262 and 12/896,525.

Turning to FIG. 3C, an exemplary embodiment of a metabolic signal module 276 is shown. The metabolic signal module 276 can comprise at least one of a lactic acid signal unit 3110 capable of providing at least one signal relating to lactic acid content in the patient's blood and/or a tissue; or a potassium signal unit 3112 capable of providing at least one signal relating to a potassium content in the patient's blood or tissue.

The metabolic signal module 276 can also comprise a metabolic signal processing unit 3130. The metabolic signal processing unit 3130 can perform any filtering, noise reduction, amplification, or other appropriate processing of the data received by the signal units 3110-3112 desired prior to calculation of the metabolic signal.

The metabolic signal module 276 can also comprise a metabolic signal calculation unit 3140. The metabolic signal calculation unit 3140 can calculate a metabolic signal from the data passed by the metabolic signal processing unit 3130. A calculated metabolic signal herein refers to any signal derivable from the provided signals.

For example, the metabolic signal calculation unit 3140 may calculate a lactic acid signal, such as may be determinable from signals yielded by a blood lactic acid sensor, as received by the lactic acid signal unit 3110 and, optionally, further processed by metabolic data processing unit 3130.

For another example, the metabolic signal calculation unit 3140 may calculate a potassium signal, such as the potassium ion content of the patient's blood. The signal may be received by potassium signal unit 3112, and, optionally, further processed by metabolic data processing unit 3130.

Turning to FIG. 3D, an exemplary embodiment of an endocrine signal module 278 is shown. The endocrine signal module 278 can comprise at least one of a prolactin signal unit 3210 capable of providing at least one signal relating to prolactin content in the patient's blood; a luteinizing hormone signal unit 3211 capable of providing at least one signal relating to luteinizing hormone content in the patient's blood; an FSH signal unit 3212 capable of providing at least one signal relating to FSH content in the patient's blood; a growth hormone signal unit 3213 capable of providing at least one signal relating to growth hormone content in the patient's blood; an ACTH signal unit 3214 capable of providing at least one signal relating to ACTH content in the patient's blood; a cortisol signal unit 3215 capable of providing at least one signal relating to cortisol content in the patient's blood; a vasopressin signal unit 3216 capable of providing at least one signal relating to vasopressin content in the patient's blood; a β-endorphin signal unit 3217 capable of providing at least one signal relating to β-endorphin content in the patient's blood; a β-lipotropin signal unit 3218 capable of providing at least one signal relating to β-lipotropin content in the patient's blood; a CRF signal unit 3219 capable of providing at least one signal relating to CRF content in the patient's blood; or another endocrine signal unit 3220 capable of providing at least one signal relating to another endocrine property.

The endocrine signal module 278 can also comprise an endocrine signal processing unit 3230. The endocrine signal processing unit 3230 can perform any filtering, noise reduction, amplification, or other appropriate processing of the data received by the signal units 3210-3220 desired prior to calculation of the endocrine signal.

The endocrine signal module 278 can also comprise an endocrine signal calculation unit 3240. The endocrine signal calculation unit 3240 can calculate an endocrine signal from the data passed by the endocrine signal processing unit 3230. A calculated endocrine signal herein refers to any signal derivable from the provided signals.

For example, the endocrine signal calculation unit 3240 may calculate a cortisol signal, such as may be determinable from signals yielded by a blood cortisol sensor, as received by the cortisol signal unit 3215 and, optionally, further processed by endocrine data processing unit 3230.

Turning to FIG. 3E, an exemplary embodiment of a stress marker signal module 279 is shown. The stress marker signal module 279 can comprise at least one of a reactive oxygen signal unit 3310 capable of providing at least one signal relating to the content of at least one reactive oxygen species in the patient's blood or a tissue; a reactive nitrogen signal unit 3311 capable of providing at least one signal relating to the content of at least one reactive nitrogen species in the patient's blood or a tissue; a catecholamine signal unit 3312 capable of providing at least one signal relating to catecholamine content in the patient's blood or a tissue; or another stress marker signal unit 3313 capable of providing at least one signal relating to another stress marker.

The stress marker signal module 279 can also comprise a stress marker signal processing unit 3330. The stress marker signal processing unit 3330 can perform any filtering, noise reduction, amplification, or other appropriate processing of the data received by the signal units 3310-3313 desired prior to calculation of the stress marker signal.

The stress marker signal module 279 can also comprise a stress marker signal calculation unit 3340. The stress marker signal calculation unit 3340 can calculate a stress marker signal from the data passed by the stress marker signal processing unit 3330. A calculated stress marker signal herein refers to any signal derivable from the provided signals.

For example, the stress marker signal calculation unit 3340 may calculate a reactive oxygen signal, such as may be determinable from signals yielded by a sensor of peroxide and/or superoxide ions in the patient's blood, as may be received by the reactive oxygen signal unit 3310 and, optionally, further processed by stress marker data processing unit 3330.

From any one or more signal(s) calculated by any calculation unit depicted in FIGS. 3A-3E, one or more autonomic activities, neurologic activities, metabolic activities, endocrine activities, or stress marker activities may be determined. Turning to FIG. 3F, a block diagram of classification unit 286 is depicted. The classification unit 286 comprises a calculated signal receiving unit 3610 capable of receiving data indicative of a calculated signal from one or more of the autonomic signal module 265, the neurologic signal module 275, the metabolic signal module 276, the endocrine signal module 278, the stress marker signal module 279, or a memory 217 storing prior outputs of such a module, and seizure classification unit 3620 capable of determining from the received data whether a seizure is epileptic, non-epileptic, another type of behavior or motor changes (e.g., behavior changes arising from dissociation or motor changes arising from Huntington's chorea or paroxysmal choreo-athetosis, or unclassifiable. The seizure classification unit 3620 may implement any appropriate algorithm(s) for classifying a seizure from at least one autonomic signal, neurologic signal, metabolic signal, endocrine signal, or stress marker signal.

The classification unit 286 may be configured to classify the seizure as a non-epileptic seizure, based on at least one of the following: an autonomic activity being not indicative of an epileptic seizure, a neurologic activity being not indicative of an epileptic seizure, a metabolic activity being not indicative of an epileptic seizure, an endocrine activity being not indicative of an epileptic seizure, or a stress marker activity being not indicative of an epileptic seizure.

In the embodiment shown in FIG. 3F, the classification unit 286 may further comprise a seizure quantification unit 3630 capable of quantifying from the received data one or more quantifiable characteristics of one or both of an epileptic seizure and a non-epileptic seizure. Exemplary quantifiable characteristics include, but are not limited to, event severity, event duration, duration of stages of the event, or values and/or ranges thereof of one or more body signals (e.g., a blood chemical value, a lactic acid content, a parameter of a motor activity, etc.), among others.

The classification unit 286 may send the output of the seizure classification unit 3620 to one or more other units or modules of the medical device 200 and/or one or more external units. The one or more other modules may then store the output, report the output to the patient, a physician, and/or a caregiver; warn the patient or a caregiver that the event under consideration is non-epileptic, etc.

The medical device system of one embodiment of the present disclosure provides for software module(s) that are capable of acquiring, storing, and processing various forms of data, such as patient data/parameters (e.g., physiological data, side-effects data, heart rate data, breathing rate data, brain-activity parameters, disease progression or regression data, quality of life data, etc.) and therapy parameter data. Therapy parameters in the case of epileptic seizures may include, but are not limited to, electrical signal parameters (e.g., frequency, pulse width, wave shape, polarity, geometry of electrical fields, on-time, off-time, etc.) that define therapeutic electrical signals delivered by the medical device in response to the detection of the seizure, medication type, dose, or other parameters, and/or any other therapeutic treatment parameter.

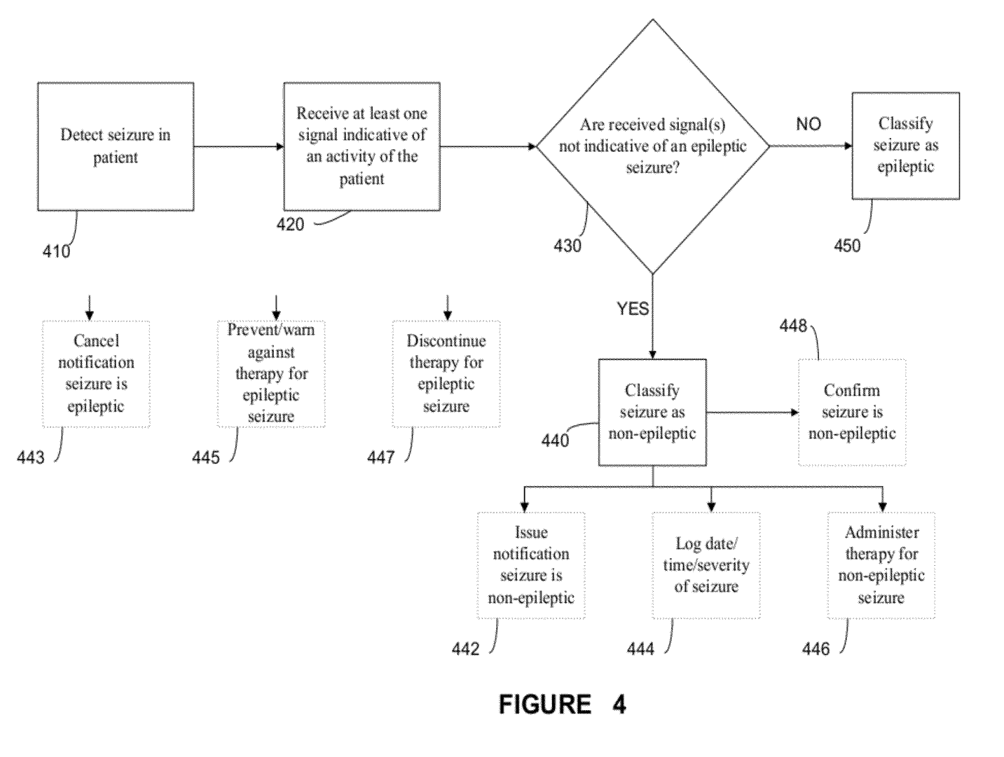

In one embodiment, the medical device 200 or an external unit 270 may also be capable of detecting a manual input from the patient. The manual input may include a magnetic signal input, a tap input, a button, dial, or switch input, a touchscreen input, a wireless data input to the medical device 200, etc. The manual input may be used to allow capture of the patient's subjective assessment of his or her epileptic events. For example, the medical device 200 may comprise one or more physical or virtual (e.g., touchscreen-implemented) buttons accessible to the patient's fingers or a caregiver's, through which the patient or caregiver may indicate he or she is having an epileptic event or is not having an epileptic event. Alternatively or in addition, the manual input may be used to gauge the patient's responsiveness. Turning now to FIG. 4, a flowchart depiction of a method according to one illustrative embodiment of the present disclosure is presented. A seizure in a patient may be detected at 410 using any appropriate technique(s), such as those described above. In one embodiment, detecting at 410 may comprise observing at least one abnormal motor activity of the patient (i.e., motor activity associated with an epileptic seizure).

At least one signal indicative of an activity of the patient may be received at 420. The at least one signal may be any one or more of the following: an autonomic signal indicative of an autonomic activity of the patient, a neurologic signal indicative of a neurologic activity of the patient, a metabolic signal indicative of a metabolic activity of the patient, an endocrine signal indicative of an endocrine activity of the patient, or a stress marker signal indicative of a stress marker of the patient, among others.

Based on the at least one received signal, the seizure may be classified at 430 as a non-epileptic seizure based on at least one of the following: the autonomic activity being an autonomic activity not indicative of an epileptic seizure, the neurologic activity being a neurologic activity not indicative of an epileptic seizure, the metabolic activity being a metabolic activity not indicative of an epileptic seizure, the endocrine activity being an endocrine activity not indicative of an epileptic seizure, or the stress marker activity being a stress marker activity not indicative of an epileptic seizure.

In a particular example, the autonomic activity not indicative of an epileptic generalized/convulsive seizure may be at least one of: a lack of a decrease of SaO2 value of the patient's blood after the onset of a generalized motor activity, relative to a reference SaO2 value, a lack of an increase in CO2 concentration of the patient's blood after the onset of a generalized motor activity, relative to a reference CO2 concentration, a lack of a decrease in a pH value of the patient's blood after the onset of a generalized motor activity essentially unchanged relative to a reference pH value, a lack of an increase in the patient's body temperature after the onset of a generalized motor activity during the seizure, relative to a reference temperature value, a lack of change in the patient's cardiac activity after the onset of a generalized motor activity, relative to a reference cardiac activity value, or a lack of an increase in an infrared activity of a portion of the patient's body after the onset of a generalized motor activity, relative to a reference infrared activity.

In an even more particular example, the autonomic activity not indicative of an epileptic seizure may be a lack of a decrease in a pH value of the patient's blood after the onset of a generalized motor activity essentially unchanged relative to a reference pH value.

In another particular example, the neurologic activity not indicative of an epileptic seizure is at least one of: a recurring crescendo-decrescendo pattern of a motor activity of the patient, a force of a motor activity of the patient, a range of motion of a motor activity of the patient, a velocity of a motor activity of the patient, a multi-directionality of a motor activity of the patient, a multi-planarity of a motor activity of the patient, a frequency of a motor activity of the patient, an incoherence between a first motor activity and a second motor activity of the patient, a lack of coactivation during motor activity of an agonist muscle group and an antagonist muscle group of the patient, or a pelvic motor activity of the patient that is the most prominent body movement of the patient.

Body movement is a type of motor activity. However, some motor activity may be associated with a lack of body movement; e.g., a tonic phase of a seizure is associated with a marked increase in muscle tone but is also associated with substantially no movement of the patient's body.

“Crescendo-decrescendo” is used herein to refer to a pattern of increasing amplitude of a motor activity, followed by a decreasing amplitude of the motor activity. The crescendo-decrescendo pattern may be recurrent and periodic or aperiodic.

In this context, coherence or being in phase refers to any feature of movement that is the same in terms of timing, between homologous parts of the body. Incoherence or not being in phase refers to any feature of movement that differs in such terms between hemihalves of the body.

A body movement may be considered in terms of planes in which the movement occurs. Various planes relating to human anatomy have been defined in the medical arts. These include a coronal plane (a plane dividing the body into front and rear portions), a sagittal plane (a plane dividing the body into left and right portions), and a transverse plane (a plane dividing the body into upper and lower portions).

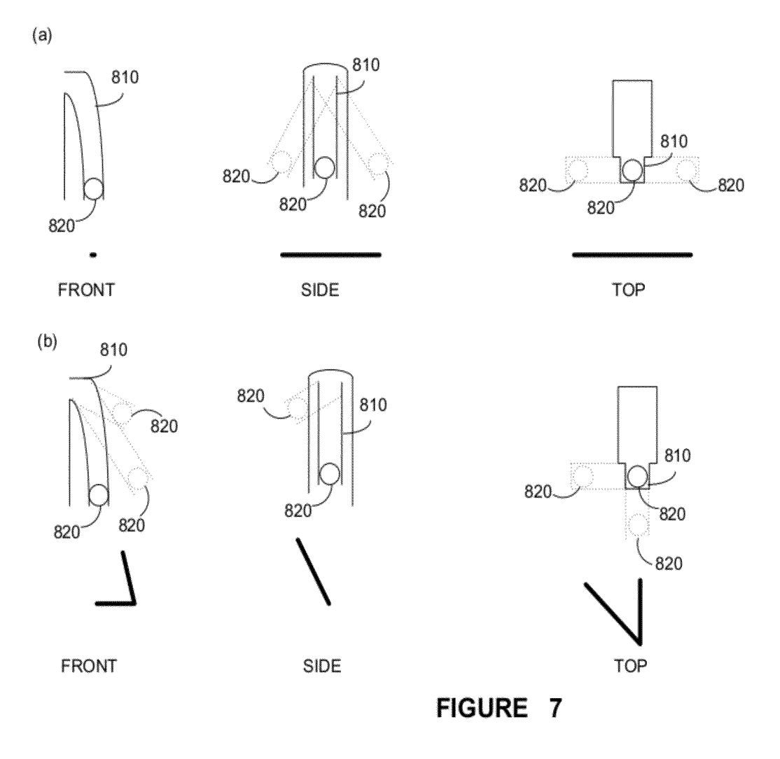

Generally speaking, epileptic movements typically are predominantly uniplanar (i.e., they typically occur substantially mainly in one body plane) and in certain joints are bi-directional (e.g. elbow flexion/extension). Non-epileptic movements are often multiplanar and/or multidirectional. FIG. 7 compares a non-limiting example of (a) a generally planar epileptic movement and (b) a generally non-planar non-epileptic movement of an arm 810, as represented by the path traced by an accelerometer 820 (e.g., disposed on a lower arm of a patient, as seen from in front of, to the side of, and above the patient.

Body movements of the same joint(s) or muscle(s)/muscle group(s) on the right and left sides of the body may be considered “synchronized” or “synchronous” if their initiation and/or termination is substantially simultaneous. In general, epileptic movements involving more than one limb or body part are more synchronous and symmetrical than non-epileptic movements. Body movements of the same joint(s) or muscle(s)/muscle group(s) on the right and left sides of the body may be considered “symmetrical” if they have substantially similar ranges of motion, velocities, amplitudes, directionalities, and/or planarities.

In a further embodiment, the neurologic activity not indicative of an epileptic seizure may further comprise at least one of: an electroencephalographic (EEG) signal not indicative of an epileptic brain activity of the patient, or a videographic motor activity analysis not indicative of an epileptic motor activity of the patient. Such further activities may assist in the classification performed at 430.

In another particular example of body signals, the metabolic activity not indicative of an epileptic seizure may be at least one of: a lack of lactic acidosis of the patient's blood sometime after the onset of a generalized motor activity during the seizure, relative to a reference lactic acid value, or a lack of a normo-kalemic metabolic acidosis of the patient's blood sometime after the onset of a generalized motor activity during the seizure, relative to a reference normo-kalemic value.

Any seizure may be subjected to the classification at 430. For example, the method of this embodiment may allow the distinction of non-epileptic generalized seizures from epileptic generalized seizures. For another example, the method of this embodiment may allow the distinction of non-epileptic partial seizures from epileptic partial seizures. The partial seizures may be simple partial or complex partial seizures.

If the seizure is to be classified as epileptic, this may be done at 450.

If the signals are not indicative of an epileptic seizure, then the seizure may be classified as non-epileptic at 440. In one embodiment, no further actions need be taken.

In one optional embodiment, the method depicted in FIG. 4 further comprises at least one of: issuing at 442 a notification that the seizure is non-epileptic, based upon classifying the seizure as non-epileptic; cancelling at 443 a notification or warning of an epileptic seizure, based upon classifying the seizure as non-epileptic; logging at 444 at least one of the seizure classification (epileptic or non-epileptic), the date of the seizure, the time of occurrence of the seizure, the severity of the seizure, the duration of the seizure, or the time between seizures; preventing or warning at 445 against delivery of a therapy for an epileptic seizure, based upon classifying the seizure as non-epileptic; administering at 446 a therapy for the non-epileptic seizure, based upon classifying the seizure is non-epileptic; or discontinuing at 447 delivery of a therapy for an epileptic seizures.

A notification that may be issued at 442 may be issued to the patient, to a caregiver, or to medical personnel. In some embodiments, the medical personnel may be an emergency medical technician or an emergency room nurse or physician. The present inventor has firsthand knowledge of a situation in which a patient presented at a rural emergency room in apparent status epilepticus, a life-threatening complication of epilepsy. A helicopter ambulance service retrieved the patient and brought him to the present inventor's current institution, a state university medical center having an advanced epilepsy care center. Upon the patient's arrival, it was rapidly determined that the patient's seizure was non-epileptic. Employment of the present method such that the rural emergency room personnel might have received a notification issued at 442 would have freed the helicopter ambulance service and the advanced epilepsy care center for patients suffering from epileptic events.

Regarding logging at 444, the date and time of occurrence of the seizure need no further discussion. Severity may be defined using any appropriate technique. In one embodiment, seizure severity may be defined from one or more of the peak intensity of the seizure, the duration of the seizure, or the extent of spread of the seizure. Intensity, duration, and extent of spread may be measured using neurologic, autonomic, metabolic, or other signals as described herein and/or in patents and applications incorporated herein by reference.

Therapies that may be administered at 446 may comprise cognitive therapy, behavioral therapy, or biofeedback. For example, the patient may receive a message that the seizure is non-epileptic, which may be followed by instructions to relax, breathe deeply, or the like. The response to one or more administered therapies may be measured (not shown) using body signals described above.

In another optional embodiment, a confirmatory notification of the non-epileptic seizure may be issued at 448. The issuance at 448 may be based on at least one of: an autonomic activity being an autonomic activity not indicative of an epileptic seizure, a neurologic activity being a neurologic activity not indicative of an epileptic seizure, a metabolic activity being a metabolic activity not indicative of an epileptic seizure, an endocrine activity being an endocrine activity not indicative of an epileptic seizure, or a stress marker activity being a stress marker activity not indicative of an epileptic seizure, wherein the activity on which the issuing is based is different from the activity on which the classifying is based.

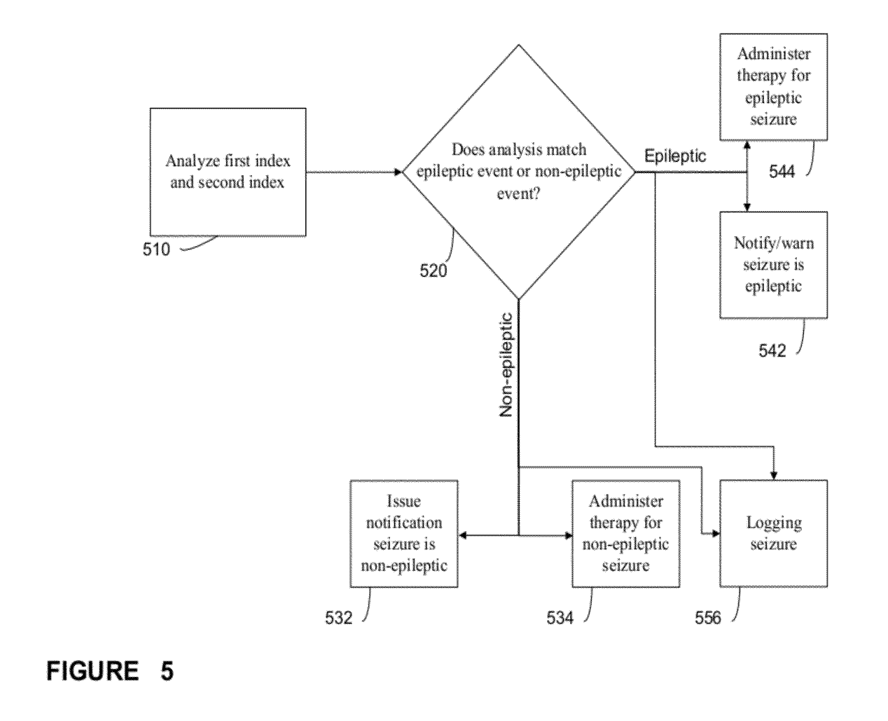

Turning to FIG. 5, a flowchart depiction of a method of distinguishing an epileptic seizure from a non-epileptic seizure is presented.

A first index and a second index, each selected from a neurologic index, an autonomic index, a metabolic index, an endocrine index, or a tissue stress marker index are analyzed at 510. The indices may be any one or more of the indices described above. In one embodiment, the analysis may comprise determining whether the first index suggests the seizure is epileptic or non-epileptic; and determining whether the second index suggests the seizure is epileptic or non-epileptic.

Thereafter, a determination may be made at 520 as to whether the analysis at 510 indicates (e.g., by matching signatures, features, values, or characteristics of indices belonging to the same class, or by cross-referencing signatures, features, values, or characteristics of indices belonging to different classes) an epileptic event or a non-epileptic event. “Indicating” here does not refer a 100% match or perfect fit/correspondence to the signature, value or characteristic. Rather, as used herein, the match is to whichever of the epileptic signature or features or the non-epileptic signature is closer to the analysis made at 510.

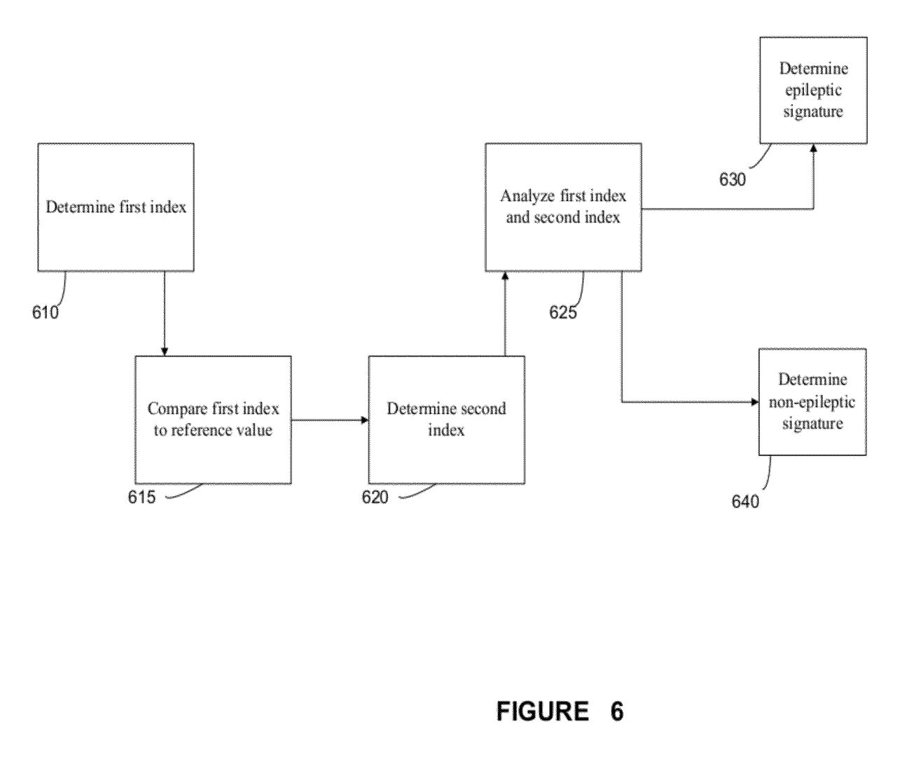

At least one further action may then be taken based on the analysis at 510. The at least one further action may be selected from: issuing at 532 a notification that the seizure is non-epileptic, based upon the analysis of the first and second index indicating that the seizure is non-epileptic; issuing at 542 a notification or warning that the seizure is epileptic, based upon the analysis of the first and second index indicating that the seizure is epileptic; administering at 534 a therapy appropriate for a non-epileptic seizure, based upon the analysis indicating that the seizure is non-epileptic; administering at 544 a therapy for an epileptic seizure, based upon the analysis indicating that the seizure is an epileptic seizure; or logging at 556 at least one of the date of the seizure, the time of occurrence of the seizure, or the severity of the seizure, which may be desirable whether the seizure is epileptic or non-epileptic. FIG. 6 presents a flowchart depiction of a method of distinguishing an epileptic seizure from a non-epileptic seizure. The method may comprise determining at 610 a first index selected from a neurologic index, an autonomic index, a metabolic index, an endocrine index, or a tissue stress marker index based on body data of a patient. In one embodiment, the first index is an autonomic index selected from a cardiac index, a respiratory index, a temperature index, an ocular index (e.g., pupillary size, rate change and hippus), a chemical index (e.g., a concentration of a chemical in a body tissue), and a blood index. In one embodiment, the first index is a neurologic index selected from a kinetic index, a cognitive index, and a neurochemical index.

The first index may be compared to a reference value, as indicated at 615. Where the index crosses or exceeds the reference value, the first index may be used to trigger collection of additional body data and/or to determine additional body indices at 620. For example, a cardiac index (or an EEG, kinetic or endocrine index) may be used to trigger determination of either additional cardiac indices or of indices belonging to other classes (e.g., metabolic, neurologic, etc.). This may include collection of, e.g., a second, third, fourth, etc. index for use in determining whether the seizure is epileptic or non-epileptic. In some embodiment, each additional index may be triggered by some or all of the existing indices that have been determined.

The indices determined at 610 and 620 may be analyzed at 625 to determine one or both of an epileptic signature, 630 or a non-epileptic signature, 640. The term “signature” is used herein to encompass values, characteristics, and other properties of the indices that are indicative of epileptic seizures or non-epileptic seizures.

The latency between the onset and termination of abnormal electrical activity in the case of epileptic seizures and the first change from an interictal baseline value varies among the different indices. Similarly, the time required for an index to return to its baseline value may vary widely. For example, the onset of abnormal/convulsive motor activity and loss of consciousness occurs nearly simultaneously with the onset of abnormal generalized brain electrical activity, while oxygen desaturation may lag behind them by a few seconds and accumulation of lactic acid will not peak for a few minutes after the appearance of abnormal kinetic/convulsive activity.

Recovery of these indices to interictal reference value does not parallel the temporal sequence of changes from inter-ictal to ictal: Oxygen saturation will recover shortly after the cessation of convulsive active while recovery of consciousness and cognitive functions and resolution of the lactic acidosis will take upwards of 30 min, in a healthy subject. The application to this disclosure of microscopic, mesoscopic and macroscopic scales is appropriate and useful as they encompass the multifarious times required for the various indices to change from interictal to ictal and from ictal to post-ictal and finally to interictal.

As previously noted, differences in the planarity of patient motions may be useful in embodiments of the present invention to distinguish between epileptic and non-epileptic movements. In many cases, generalized epileptic movements are characterized by a high level of planarity. This is not to imply that epileptic movements are completely planar, but rather that such movements may be distinguished from movements in non-epileptic seizures by comparing the movements in terms of their degree of uni-planarity, with epileptic seizures being characterized by a significantly higher level of uni-planarity. In FIG. 7(a), an accelerometer 820 worn, for example, on a lower arm of a patient, may be used to determine a kinetic index or movement score that indicates the movement is generally planar. As seen in the side and top views, the arm movement occurs in a single plane. Although not fully shown, the movement may also include flexion and/or bending at the elbow, while still remaining substantially planar.

In FIG. 7(b), in contract, as seen most clearly in the front and top views, non-epileptic seizures are typically characterized by occurring in a random, multiplanar way. A kinetic index characterizing the level of planarity of these movements would show a significantly different value from the largely planar movements characteristic of seizures and depicted in FIG. 7(a). In embodiments of the present invention, accelerometer calculations of the planarity of the movement—either alone or in conjunction with other kinetic indices such as force, magnitude of acceleration, etc.—may be used to characterize a seizure as either epileptic or non-epileptic.

The above methods may be performed by a non-transitive computer readable program storage device encoded with instructions that, when executed by a computer, perform the method described herein.

All of the methods and apparatuses disclosed and claimed herein may be made and executed without undue experimentation in light of the present disclosure. While the methods and apparatus of this invention have been described in terms of particular embodiments, it will be apparent to those skilled in the art that variations may be applied to the methods and apparatus and in the steps, or in the sequence of steps, of the method described herein without departing from the concept, spirit, and scope of the invention, as defined by the appended claims. It should be especially apparent that the principles of the invention may be applied to selected cranial nerves other than, or in addition to, the vagus nerve to achieve particular results in treating patients having epilepsy, depression, or other medical conditions.

The particular embodiments disclosed above are illustrative only as the invention may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

### REFERENCES

The following references, to the extent that they provide exemplary procedural or other details supplementary to those set forth herein, are specifically incorporated herein by reference.
U.S. patent application Ser. No. 12/756,065, filed Apr. 7, 2010
U.S. patent application Ser. No. 12/770,562, filed Apr. 29, 2010
U.S. patent application Ser. No. 12/896,525, filed Oct. 1, 2010
U.S. patent application Ser. No. 13/040,996, filed Mar. 4, 2011
U.S. patent application Ser. No. 13/091,033, filed Apr. 20, 2011
U.S. patent application Ser. No. 13/098,262, filed Apr. 29, 2011
U.S. Pat. No. 4,702,254
U.S. Pat. No. 4,867,164
U.S. Pat. No. 5,025,807
U.S. Pat. No. 6,961,618
U.S. Pat. No. 7,457,665

1. A method of distinguishing a non-epileptic seizure from an epileptic seizure in a patient, comprising:
detecting a seizure in a patient based on at least one first body signal of the patient selected from an autonomic signal, a neurologic signal, a metabolic signal, an endocrine signal, and a tissue stress marker signal;
analyzing at least one second body signal of the patient selected from an autonomic signal, a neurologic signal, a metabolic signal, an endocrine signal, and a tissue stress marker signal;
determining, based on the analyzing of the at least one second body signal, at least a first classification index comprising at least one of an epileptic seizure index and a non-epileptic seizure index; and
classifying the seizure as one of a non-epileptic seizure or an epileptic seizure based on the at least a first classification index.

2. The method of claim 1 wherein classifying the seizure as one of a non-epileptic seizure or an epileptic seizure comprises classifying the seizure as non-epileptic based on at least one of
an epileptic seizure index reaching or exceeding a threshold indicative of an epileptic seizure, or
a non-epileptic seizure index reaching or exceeding a threshold indicative of a non-epileptic seizure.

3. The method of claim 1 wherein said at least one second body signal comprises at least two body signals, each of said at least two body signals being selected from an autonomic signal, a neurologic signal, a metabolic signal, an endocrine signal, and a tissue stress marker signal.

4. The method of claim 1, wherein said at least a first classification index comprises at least one index selected from:
an SaO2 index indicating that the patient's blood oxygen saturation is above a reference value after the onset of a generalized motor activity,
a CO2 index indicating that the patient CO2 blood concentration is below a reference CO2 blood concentration after the onset of a generalized motor activity,
a pH index indicating that the pH of the patient's arterial blood is above a reference value after the onset of a generalized motor activity relative to a reference pH value,
a pH index indicating the lack of a decrease in a pH value of said patient's arterial blood after the onset of a generalized motor activity relative to a reference value,
a body temperature index indicating that the patient's body temperature is below a reference value after the onset of a generalized motor activity,
a cardiac index remaining below a reference value following onset of generalized motor activity,
an infrared index from a target portion of the patient's body remaining below a reference value after the onset of a generalized motor activity; or
a skin resistance index from a target portion of the patient's body remaining outside the range of value of a resistance index indicative of a generalized tonic, clonic, or tonic-clonic epileptic seizure.

5. The method of claim 1, wherein said at least a first classification index comprises at least one kinetic index indicative of at least one of:
at least one of a repeating, periodic, or aperiodic crescendo-decrescendo pattern of a motor activity of said patient,
a force of a motor activity that is outside epileptic seizure reference values;
a range of motion of a motor activity of said patient that is outside a range indicative of an epileptic seizure,
a velocity of a motor activity of said patient that is outside a range indicative of an epileptic seizure,
a multi-directionality of a motor activity of said patient that is outside a range of an epileptic seizure,
a multi-planarity of a motor activity of said patient that is outside a range indicative of an epileptic seizure,
a frequency of a motor activity of said patient that is outside a frequency range of motor activity of an epileptic seizure;
a phase asynchrony between a first and second homologous body portions in a patient without pre-existing motor deficit;
at least one of a difference in amplitude, force, velocity and direction of movement between homologous first and second body portions in a patient without pre-existing motor deficit having bilateral movements;
a pelvic thrust or pelvic motor activity of said patient that is the most prominent body movement of said patient.

6. The method of claim 1, wherein said at least a first classification index comprises a neurological index indicative of at least one of:
a spontaneous or evoked cortical electrical signal not indicative of epileptic brain activity of said patient; or
a videographic image signal that is not indicative of an epileptic motor activity of said patient.

7. The method of claim 1, wherein said at least a first classification index comprises at least one of a metabolic index selected from:
a lactic acid concentration in the patient's blood that remains below a reference concentration;
a lactic acid concentration in the patient's blood indicative of a lack of lactic acidosis in said patient's blood after the onset of a generalized motor activity, relative to an inter-ictal lactic acid value, or
a normal serum potassium concentration in a patient with metabolic acidosis after the onset of a generalized motor activity.

8. The method of claim 1, further comprising at least one of:
issuing a notification that the seizure is non-epileptic, based upon classifying said seizure as non-epileptic;
logging at least one of the date of the non-epileptic seizure, the time of occurrence of the non-epileptic seizure, or the severity of the non-epileptic seizure; or
administering a therapy for said non-epileptic seizure, based upon classifying said seizure as non-epileptic.

9. The method of claim 1, further comprising:
determining at least a second classification index selected from:
an autonomic index having a value or characteristic that is not indicative of an epileptic seizure,
a neurologic index having a value or characteristic that is not indicative of an epileptic seizure,
a metabolic index having a value or characteristic that is not indicative of an epileptic seizure,
an endocrine index having a value or characteristic that is not indicative of an epileptic seizure, and
a tissue stress marker index having a value or characteristic that is not indicative of an epileptic seizure,
wherein classifying said seizure as a non-epileptic seizure is based at least in part upon both said first classification index and said second classification index.

10. The method of claim 1 wherein classifying the seizure as a non-epileptic seizure based on the at least a first classification index comprises
classifying the seizure as a non-epileptic seizure only if the at least a first classification index is within non-epileptic seizure reference values.

11. A medical device system, comprising:
at least one sensor configured to receive at least one of an autonomic signal indicative of an autonomic activity of a patient, a neurologic signal indicative of a neurologic activity of said patient, a metabolic signal indicative of a metabolic activity of said patient, an endocrine signal indicative of an endocrine activity of said patient, or a tissue stress marker signal indicative of a tissue stress marker activity of said patient;
a seizure detection unit configured to detect a seizure in a patient based on said at least one autonomic, neurologic, metabolic, endocrine, or tissue stress marker signal;
at least one classification index determination unit configured to determine at least a first classification index selected from an autonomic index, a neurologic index, a metabolic index, an endocrine index, and a tissue stress marker index; and
a seizure classification unit configured to classify said epileptic seizure as one of an epileptic seizure and a non-epileptic seizure based at least in part on said at least a first classification index.

12. The medical device system of claim 11, wherein said classification index determination unit is configured to determine
at least one kinetic index indicating one of the onset or a lack of generalized motor activity; and
at least one autonomic index selected from at least one of:
an SaO2 index indicating that the patient's blood oxygen saturation remains above a reference value after said kinetic index indicates the onset of a generalized motor activity,
a CO2 index indicating that the patient's CO2 blood concentration remains below a reference CO2 blood concentration after said kinetic index indicates the onset of a generalized motor activity,
a pH index indicating that the pH of the patient's arterial blood remains above a reference value after said kinetic index indicates the onset of a generalized motor activity relative to a reference pH value,
a pH index indicating the lack of a decrease in a pH value of said patient's arterial blood after said kinetic index indicates the onset of a generalized motor activity relative to a reference value;
a body temperature index indicating that the patient's body temperature remains below a reference value after said kinetic index indicates the onset of a generalized motor activity,
a cardiac index indicating that cardiac indices are below a reference value after said kinetic index indicates the onset of generalized motor activity, or
an infrared index indicating that infrared radiation from a target portion of the patient's body remains below a reference value after said kinetic index indicates the onset of a generalized motor activity.

13. The medical device system of claim 11 wherein said at least a first classification index is a neurological index comprising a kinetic index indicative of at least one of:
a direction of movement of a target portion of the patient's body in one plane, indicative of an epileptic seizure;
a force of a motor activity of said patient that exceeds an epileptic seizure reference value,
a range of motion of a motor activity of said patient that is within a range indicative of an epileptic seizure,
a velocity of a movement of a target portion of the patient's body that is indicative of an epileptic seizure,
a degree of synchronization or symmetry between movement of a right portion of the patient's body and a left portion of the patient's body indicative of an epileptic seizure;
a frequency of a movement of said patient that is indicative of an epileptic seizure;
a joint position that is indicative of an epileptic seizure;
a body posture that is indicative of an epileptic seizure;
a repeating, periodic or aperiodic crescendo-decrescendo pattern of a motor activity of said patient characteristic of a non-epileptic seizure,
a velocity of a motor activity of said patient that is outside a range indicative of an epileptic seizure,
a range of motion of a motor activity of said patient that is indicative of a non-epileptic seizure,
a velocity of a movement of a target portion of the patient's body that is indicative of a non-epileptic seizure;
a force of a motor activity that is indicative of a non-epileptic seizure;
a multi-directionality of a motor activity of said patient that is indicative of a non-epileptic seizure,
a multi-planarity of a motor activity of said patient that is indicative of a non-epileptic seizure,
a frequency of a movement of a target portion of the patient's body that is indicative of a non-epileptic seizure;
a degree of synchronization or symmetry between movement of a right portion of the patient's body and a left portion of the patient's body indicative of a non-epileptic seizure;
a joint position that is indicative of an epileptic seizure;
a body posture that is indicative of an epileptic seizure;
or
a pelvic thrust or pelvic motor activity of said patient that is the most prominent body movement of said patient and is indicative of a non-epileptic seizure.

14. The medical device system of claim 11, wherein said at least a first classification index is at least one metabolic index selected from:
a lactic acid concentration in the patient's blood that remains below a reference concentration;
a lactic acid concentration in the patient's blood indicative of a lack of lactic acidosis in said patient's blood after the onset of a generalized motor activity, relative to an inter-ictal lactic acid value, and
a normal potassium concentration in a patient with metabolic acidosis after the onset of a generalized motor activity.

15. The medical device system of claim 11, further comprising a therapy unit configured to provide at least one response to said classification unit classifying a detected seizure as a non-epileptic seizure, wherein said at least one response is selected from
administering a therapy appropriate for a non-epileptic seizure,
discontinuing delivery of a therapy for epileptic seizures,
preventing delivery of a therapy for epileptic seizures,
warning against delivery of a therapy for epileptic seizure,
cancelling a warning for an epileptic seizure;
providing a notification that the seizure is a non-epileptic seizure; or
logging the seizure as non-epileptic along with at least one of the date, time of occurrence, duration, or intensity of the non-epileptic seizure.

16. The medical device system of claim 11, further comprising a notification unit configured to notify at least one of said patient, a caregiver, or a medical professional that said seizure is non-epileptic, based upon said classification unit classifying a detected seizure as a non-epileptic seizure.

17. A method of distinguishing an epileptic seizure from a non-epileptic seizure, comprising:
identifying an unclassified seizure;
determining a first seizure classification index having an index class selected from a neurologic index class, an autonomic index class, a motor index class, a tissue stress marker index class, or a metabolic index class;
determining a second seizure classification index having an index class selected from a neurologic index class, an autonomic index class, a motor index class, a tissue stress marker index class, or a metabolic index class;
classifying said seizure as one of an epileptic seizure or a non-epileptic seizure based on both said first and said second seizure classification indices; and
taking at least one further action based on said classifying, wherein said at least one further action is selected from:
issuing a notification that the seizure is non-epileptic;
issuing a notification that the seizure is epileptic;
administering a therapy for a non-epileptic seizure;
administering a therapy for an epileptic seizure; or
logging at least one of whether the seizure is an epileptic or non-epileptic seizure and at least one of the date of the seizure, the time of occurrence of the seizure, the severity of the seizure, the time elapsed from a previous seizure and the frequency per unit time of the same type of seizure.

18. The method of claim 17 wherein said classifying comprises determining if said first and second seizure classification indices match one of a signature of an epileptic seizure, a pattern of an epileptic seizure, a characteristic of an epileptic seizures, a signature of a non-epileptic seizure, a pattern of a non-epileptic seizure, and a characteristic of a non-epileptic seizure.

19. A method, comprising:
receiving a kinetic signal from at least one target of the patient's body;
determining at least one kinetic index based on said kinetic signal;
identifying an unclassified seizure based on the at least one kinetic index;
receiving at least one of a non-kinetic neurologic index and an autonomic index; and
classifying the seizure as an epileptic seizure or non-epileptic seizure based on the at least one of a non-kinetic neurologic index and an autonomic index.
